(19) United States
(12) Patent Application Publication
(10) Pub. No.: US 2007/0185148 A1
(43) Pub. Date: Aug. 9, 2007

(54) M3 muscarinic acetylchoine receptor antagonists

(75) Inventors: Jakob Busch-Petersen, King of Prussia, PA (US); Anthony W. J. Cooper, Hertfordshire (GB); Dramane I. Laine, King of Prussia, PA (US); Michael R. Palovich, King of Prussia, PA (US); Zehong Wan, King of Prussia, PA (US); Hongxing Yan, King of Prussia, PA (US); Chongjie Zhu, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, Greenford, Middlesex (GB)

(21) Appl. No.: 10/598,885

(22) PCT Filed: Mar. 17, 2004
(86) PCT No.: PCT/US04/08025

§ 371 Date: Sep. 14, 2006

(51) Int. Cl. A61K 31/4745; C07D 451/02

(52) U.S. Cl. 514/294; 546/94

(57) ABSTRACT

Muscarinic Acetylcholine receptor antagonists and methods of using them are provided.

### FIELD OF THE INVENTION

This invention relates to novel bicyclic amine compounds, pharmaceutical compositions, processes for their preparation, and use thereof in treating M₃ muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs, and these receptors can mediate both inhibitory and excitatory actions. For example, in smooth muscle found in the airways, bladder and gastrointestinal tract, M₃ mAChRs mediate contractile responses (1989. The Muscarinic Receptors. The Humana Press, Inc., Clifton, N.J.).

Muscarinic acetylcholine receptor dysfunction has been noted in a variety of different pathophysiological states. For instance, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation. This mAChR dysfunction results in airway hyperreactivity mediated by increased stimulation of M₃ mAChRs. Similarly, inflammation of the gastrointestinal tract in inflammatory bowel disease (IBD) results in M₃ mAChR-mediated hypermotility (Oprins, J. C. J., HP. Meijer, and J. A. Groot. 2000. Tumor Necrosis Factor-{alpha} Potentiates Ion Secretion Induced by Muscarinic Receptor Activation in the Human Intestinal Epithelial Cell Line HT29cl.19A. Ann NY Acad Sci 915:102-106). Incontinence due to bladder hypercontractility has also been demonstrated to be mediated through increased stimulation of M₃ mAChRs. Thus the identification of subtype-selective mAChR antagonists may be useful as therapeutics in these mAChR-mediated diseases.

Despite the large body of evidence supporting the use of anti-muscarinic receptor therapy for treatment of a variety of disease states, relatively few anti-muscarinic compounds are in use in the clinic. Thus, there remains a need for novel compounds that are capable of causing blockade at M₃ mAChRs. Conditions associated with an increase in stimulation of M₃ mAChRs, such as asthma, COPD, IBD and urinary incontinence would benefit by compounds that are inhibitors of mAChR binding.

### SUMMARY OF THE INVENTION

This invention provides for a method of treating a muscarinic acetylcholine receptor (mAChR) mediated disease, wherein acetylcholine binds to an M₃ mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent: 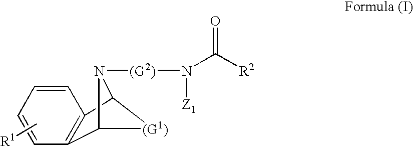
wherein:

Z1 is, independently, H or C₁₋₆ alkyl;

R¹ is, independently, a substituent selected from the group consisting of: Hydrogen, halogen, C₁₋₄ alkyl, —C(O)(C₁₋₆ alkyl), —CO₂(C₁₋₆ alkyl), —C(O)(aryl) and —C(O)[(C₁₋₆ alkyl)-aryl];

G¹ is, independently, CH₂—CH₂ or CH═CH;

G² is, independently, C₄₋₇alkyl or a group of the formula (a), (b) or (c): 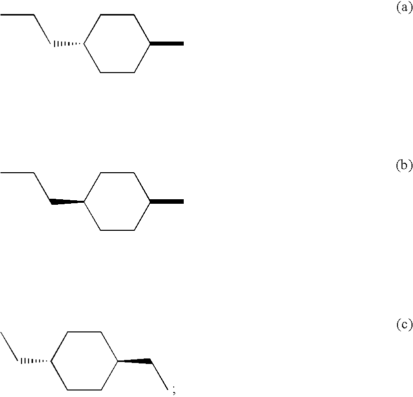
R² is, independently, a group of the formula (d) or (e): 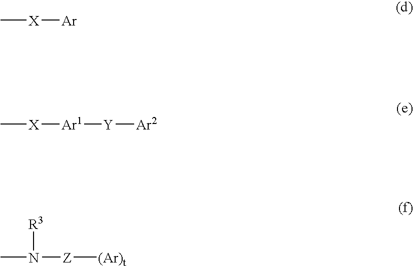
wherein

X is, independently, a bond, NR³ or C₁₋₄ alkyl;

R³ is, independently, selected form the group consisting of H, optionally substituted C₁₋₆ alkyl and C₁₋₄ alkyl-aryl;

Z is, independently, optionally substituted C₁₋₆ alkyl, and C₁₋₆ alkyl-Y²; or Z and R³ or Z and Ar may come together to form a 4-7 membered ring;

Ar is selected from the group consisting of an optionally substituted phenyl ring, an optionally substituted 5- or 6-membered aromatic heterocyclic ring; an optionally substituted bicyclic or heterobicyclic ring system; and an optionally substituted tricyclic or heterotricyclic ring system;

Ar¹ and Ar², are each, independently, selected from the group consisting of an optionally substituted phenyl ring and an optionally substituted 5- or 6-membered aromatic heterocyclic ring;

Y is, independently, selected from the group consisting of a bond, —NHCO—, —CONH—, —CH₂—, and —(CH₂)ₘY¹(CH₂)ₙ— wherein Y¹ represents O, S, SO₂, or CO and m and n each represent zero or 1 such that the sum of m+n is zero and 1; provided that when R² represents a group of formula (d) wherein X is a bond, any substituent present in Ar ortho to the carboxamide moiety is necessarily a hydrogen or a methoxy group

Y² is, independently, selected from the group consisting of NR³, O, S, —NHC(O)—, and —C(O)NH—;

t is, independently, selected from the group consisting of an integer between 0 and 3.

When R¹ represents an aroyl, or aroylC₁₋₄alkyl, the aryl moiety may be selected from an optionally substituted phenyl ring or an optionally substituted 5- or 6-membered heterocyclic ring. In the group R¹ an aryl moiety may be optionally substituted by one or more substituents selected from hydrogen, halogen, amino, cyano, C₁₋₄alkyl, C₁₋₄alkylamino, C₁₋₄dialkylamino, C₁₋₄alkylamido, C₁₋₄alkanoyl, or R⁵R⁶NCO where each of R⁵ and R⁶ independently represents a hydrogen atom or C₁₋₄alkyl group.

A halogen atom present in the compounds of formula (I) may be fluorine, chlorine, bromine or iodine.

An optionally substituted 5- or 6-membered heterocyclic aromatic ring, as defined for any of the groups Ar, Ar¹ or Ar² may contain from 1 to 4 heteroatoms selected from O, N or S. When the ring contains 2-4 heteroatoms, one is preferably selected from O, N and S and the remaining heteroatoms are preferably N. Examples of 5 and 6-membered heterocyclic groups include furyl, thienyl, pyrrolyl, oxazolyl, thiazolyl, imidazolyl, oxadiazolyl, thiadiazolyl, pyridyl, triazolyl, triazinyl, pyridazyl, pyrimidinyl, pyrazolyl, isothiazolyl, and isoxazolyl.

Examples of bicyclic, for example bicyclic aromatic or heteroaromatic, ring systems for Ar include naphthyl, indazolyl, indolyl, benzofuranyl, benzothienyl, benzothiazolyl, benzimidazolyl, benzoxazolyl, benzisoxazolyl, benzisothiazolyl, quinolinyl, quinoxolinyl, quinazolinyl, cinnolinyl, isoquinolinyl, pyrazolo[1,5-a]pyrimidyl, pyrrolo[3,2-b]pyridyl, pyrrolo[3,2-c]pyridyl, thieno[3,2-b]thiophenyl, 1,2-dihydro-2-oxo-quinolinyl, 3,4-dihydro-3-oxo-2H-benzoxazinyl, 1,2-dihydro-2-oxo-3H-indolyl.

The rings Ar, Ar¹, or Ar² may each independently be substituted optionally by one or more substituents selected from: a hydrogen or halogen atom, or a hydroxy, oxo, cyano, nitro, trifluoromethyl, C₁₋₄alkyl, C₁₋₄alkoxy, C₁₋₄alkylenedioxy, C₁₋₄alkanoyl, C₁₋₄alkylsulfonyl, C₁₋₄alkylsulfinyl, C₁₋₄alkylthio, R⁷SO₂N(R⁸)—, R⁷R⁸NSO₂—, R⁷R⁸N—, R⁷R⁸NCO—, R⁷OC(O)— or R⁷CON(R⁸)— group wherein each of R⁷ and R⁸ independently represents a hydrogen atom or a C₁₋₄ alkyl group, or R⁷R⁸ together form a C₃₋₆ alkylene chain.

Alternatively, Ar and Ar² may be optionally substituted by one or more 5- or 6-membered heterocyclic rings, as defined above, optionally substituted by a C₁₋₂ alkyl or R⁷R⁸N— group; wherein R⁷ and R⁸ are as defined above.

In the rings Ar and Ar² substituents positioned ortho to one another may be linked to form a 5- or 6-membered ring.

It will be appreciated that for use in medicine the salts of formula (I) should be physiologically acceptable. Suitable physiologically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Other non-physiologically acceptable salts eg. oxalates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

The following terms, as used herein, refer to:

“optionally substituted”—one or more substituents selected from: halo; hydroxy; hydroxy substituted C₁₋₁₀alkyl; C₁₋₁₀ alkoxy, such as methoxy or ethoxy; S(O)_{m′}C₁₋₁₀ alkyl, wherein m′ is 0, 1 or 2, such as methyl thio, methyl sulfinyl or methyl sulfonyl; amino, mono & di-substituted amino, such as in the NR₄R₅ group; NHC(O)R₄; C(O)NR₄R₅; C(O)OH; C(O)OR⁴S(O)₂NR⁴R⁵; NHS(O)₂R₂₀, C₁₋₁₀ alkyl, such as methyl, ethyl, propyl, isopropyl, or t-butyl; halosubstituted C₁₋₁₀ alkyl, such CF₃;
“halo”—all halogens, that is chloro, fluoro, bromo and iodo.
“C₁₋₁₀alkyl” or “alkyl”—both straight and branched chain moieties of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like.
“cycloalkyl” is used herein to mean cyclic moiety, preferably of 3 to 8 carbons, including but not limited to cyclopropyl, cyclopentyl, cyclohexyl, and the like.
“alkenyl” is used herein at all occurrences to mean straight or branched chain moiety of 2-10 carbon atoms, unless the chain length is limited thereto, including, but not limited to ethenyl, 1-propenyl, 2-propenyl, 2-methyl-1-propenyl, 1-butenyl, 2-butenyl and the like.
“aryl”—phenyl and naphthyl;
“heteroaryl” (on its own or in any combination, such as “heteroaryloxy”, or “heteroaryl alkyl”)—a 5-10 membered aromatic ring system in which one or more rings contain one or more heteroatoms selected from the group consisting of N, O or S, such as, but not limited, to pyrrole, pyrazole, furan, thiophene, quinoline, isoquinoline, quinazolinyl, pyridine, pyrimidine, oxazole, tetrazole, thiazole, thiadiazole, triazole, imidazole, or benzimidazole.
“heterocyclic” (on its own or in any combination, such as “heterocyclicalkyl”)—a saturated or partially unsaturated 4-10 membered ring system in which one or more rings contain one or more heteroatoms selected from the group consisting of N, O, or S; such as, but not limited to, pyrrolidine, piperidine, piperazine, morpholine, tetrahydropyran, thiomorpholine, or imidazolidine. Furthermore, sulfur may be optionally oxidized to the sulfone or the sulfoxide.
“arylalkyl” or “heteroarylalkyl” or “heterocyclicalkyl” is used herein to mean C₁₋₁₀ alkyl, as defined above, attached to an aryl, heteroaryl or heterocyclic moiety, as also defined herein, unless otherwise indicated.

Particular preferred compounds according to the invention include those specifically exemplified and named hereinafter:
2-Methyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
8-Chloro-2-methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
8-Methyl-quinoline-5-carboxylic acid (4-{2-[6-butyryl-1,2,3,4-tetrahydro-1,4-epiazano-naphthalen-9-yl]-ethyl}-cyclohexyl)-amide;
2-Methyl-quinoline-5-carboxylic acid (trans-4-{2-[6-butyryl-1,2,3,4-tetrahydro-1,4-epiazano-naphthalen-9-yl]-ethyl}-cyclohexyl)-amide;
2-Methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
N′-(trans-4-[2-(1,2,3,4-tetrahydro-1,4-epiazano-naphthalen-9-yl)-ethyl]-cyclohexyl)-1-(2-phenylethyl)-1-(phenylmethyl)urea;
1-(trans-4-[2-(1,2,3,4-tetrahydro-1,4-epiazano-naphthalen-9-yl)-ethyl]-cyclohexyl)-3-(2-hydroxy-2,2-diphenylethyl)urea;
N-[2-({[(trans-4-[2-(1,2,3,4-tetrahydro-1,4-epiazano-naphthalen-9-yl)-ethyl]-cyclohexyl)amino]carbonyl}amino)ethyl]-4-methylbenzenesulfonamide;
1,1-dimethylethyl N-{[(trans-4-[2-(1,2,3,4-tetrahydro-1,4-epiazano-naphthalen-9-yl)-ethyl]-cyclohexyl)amino]carbonyl}-L-phenylalaninate;
N′-(trans-4-[2-(1,2,3,4-tetrahydro-1,4-epiazano-naphthalen-9-yl)-ethyl]-cyclohexyl)-N-(3,3-diphenylpropyl)-N-methylurea;
3-[({[(trans-4-[2-(1,2,3,4-tetrahydro-1,4-epiazano-naphthalen-9-yl)-ethyl]-cyclohexyl)amino]carbonyl}amino)methyl]benzenesulfonamide formate;
1-(trans-4-[2-(1,2,3,4-tetrahydro-1,4-epiazano-naphthalen-9-yl)-ethyl]-cyclohexyl)-3-(2-hydroxy-1,1-diphenylethyl)urea formate;
N,N′-bis(trans-4-[2-(1,2,3,4-tetrahydro-1,4-epiazano-naphthalen-9-yl)-ethyl]-cyclohexyl)urea
1-(trans-4-[2-(1,2,3,4-tetrahydro-1,4-epiazano-naphthalen-9-yl)-ethyl]-cyclohexyl)-3-(3-hydroxy-3,3-diphenylpropyl)urea formate;
1-(trans-4-[2-(1,2,3,4-tetrahydro-1,4-epiazano-naphthalen-9-yl)-ethyl]-cyclohexyl)-3-[(1S)-2-hydroxy-1-methyl-2,2-diphenylethyl]urea formate;
1-(trans-4-[2-(1,2,3,4-tetrahydro-1,4-epiazano-naphthalen-9-yl)-ethyl]-cyclohexyl)-3-(cyclohexylmethyl)urea;
1-(trans-4-[2-(1,2,3,4-tetrahydro-1,4-epiazano-naphthalen-9-yl)-ethyl]-cyclohexyl)-3-[(2-hydroxyphenyl)methyl]urea;
1-(trans-4-[2-(1,2,3,4-tetrahydro-1,4-epiazano-naphthalen-9-yl)-ethyl]-cyclohexyl)-3-[2-(1-methyl-1H-pyrrol-2-yl)ethyl]urea;
1-(trans-4-[2-(1,2,3,4-tetrahydro-1,4-epiazano-naphthalen-9-yl)-ethyl]-cyclohexyl)-3-[(4-fluorophenyl)methyl]urea;
1-(trans-4-[2-(1,2,3,4-tetrahydro-1,4-epiazano-naphthalen-9-yl)-ethyl]-cyclohexyl)-3-[(4-fluorophenyl)methyl]urea;
1-(trans-4-[2-(1,2,3,4-tetrahydro-1,4-epiazano-naphthalen-9-yl)-ethyl]-cyclohexyl)-3-(2,3-dihydro-1H-inden-1-yl)urea;
1-(trans-4-[2-(1,2,3,4-tetrahydro-1,4-epiazano-naphthalen-9-yl)-ethyl]-cyclohexyl)-3-(3-phenylpropyl)urea;
1-(trans-4-[2-(1,2,3,4-tetrahydro-1,4-epiazano-naphthalen-9-yl)-ethyl]-cyclohexyl)-3-[(4-chlorophenyl)methyl]urea;
1-(trans-4-[2-(1,2,3,4-tetrahydro-1,4-epiazano-naphthalen-9-yl)-ethyl]-cyclohexyl)-3-(1,1a,6,6a-tetrahydrocyclopropa[a]inden-1-yl)urea;
N′-(trans-4-[2-(1,2,3,4-tetrahydro-1,4-epiazano-naphthalen-9-yl)-ethyl]-cyclohexyl)-N-(3-hydroxypropyl)-N-(phenylmethyl)urea;
1-(trans-4-[2-(1,2,3,4-tetrahydro-1,4-epiazano-naphthalen-9-yl)-ethyl]-cyclohexyl)-3-{[4-(trifluoromethyl)phenyl]methyl}urea;
1,1-dimethylethyl 2-{[(trans-4-[2-(1,2,3,4-tetrahydro-1,4-epiazano-naphthalen-9-yl)-ethyl]-cyclohexyl)amino]carbonyl}benzoate;
N-(trans-4-[2-(1,2,3,4-tetrahydro-1,4-epiazano-naphthalen-9-yl)-ethyl]-cyclohexyl)-2,2-diphenylpropanamide;
N-(trans-4-[2-(1,2,3,4-tetrahydro-1,4-epiazano-naphthalen-9-yl)-ethyl]-cyclohexyl)-4-[(phenylcarbonyl)amino]benzamide;
1-(trans-4-[2-(1,2,3,4-tetrahydro-1,4-epiazano-naphthalen-9-yl)-ethyl]-cyclohexyl)-3-(2,2-diphenylethyl)urea;
N′-(trans-4-[2-(1,2,3,4-tetrahydro-1,4-epiazano-naphthalen-9-yl)-ethyl]-cyclohexyl)-N,1-bis(phenylmethyl)urea;
1-(trans-4-[2-(1,2,3,4-tetrahydro-1,4-epiazano-naphthalen-9-yl)-ethyl]-cyclohexyl)-3-(3,3-diphenylpropyl)urea;
1-Benzyl-3-{4-[2-(1,2,3,4-tetrahydro-1,4-epiazano-naphthalen-9-yl)-ethyl]-cyclohexyl}-urea
1-(1-Naphthalen-1-yl-ethyl)-3-{4-[2-(1,2,3,4-tetrahydro-1,4-epiazano-naphthalen-9-yl)-ethyl]-cyclohexyl}-urea.
   Preferred compounds according to the invention include those specifically exemplified and named hereinafter:
1-(trans-4-[2-(1,2,3,4-tetrahydro-1,4-epiazano-naphthalen-9-yl)-ethyl]-cyclohexyl)-2-(4-pyridinyl)acetamide;
1-(trans-4-[2-(1,2,3,4-tetrahydro-1,4-epiazano-naphthalen-9-yl)-ethyl]-cyclohexyl)-3-(2-pyridinylmethyl)urea;
1-(trans-4-[2-(1,2,3,4-tetrahydro-1,4-epiazano-naphthalen-9-yl)-ethyl]-cyclohexyl)-3-(4-hydroxycyclohexyl)urea;
N′-(trans-4-[2-(1,2,3,4-tetrahydro-1,4-epiazano-naphthalen-9-yl)-ethyl]-cyclohexyl)-N-methyl-N-(phenylmethyl)urea;
1-(trans-4-[2-(1,2,3,4-tetrahydro-1,4-epiazano-naphthalen-9-yl)-ethyl]-cyclohexyl)-3-[2-(2-pyridinyl)ethyl]urea;
N-(trans-4-[2-(1,2,3,4-tetrahydro-1,4-epiazano-naphthalen-9-yl)-ethyl]-cyclohexyl)-2-(2-pyrimidinylthio)acetamide;
N-(trans-4-[2-(1,2,3,4-tetrahydro-1,4-epiazano-naphthalen-9-yl)-ethyl]-cyclohexyl)-3-quinolinecarboxamide;
N-(trans-4-[2-(1,2,3,4-tetrahydro-1,4-epiazano-naphthalen-9-yl)-ethyl]-cyclohexyl)-1-methyl-1H-indole-2-carboxamide;
E)-N-(trans-4-[2-(1,2,3,4-tetrahydro-1,4-epiazano-naphthalen-9-yl)-ethyl]-cyclohexyl)-4-oxo-4-phenyl-2-butenamide;
1-(trans-4-[2-(1,2,3,4-tetrahydro-1,4-epiazano-naphthalen-9-yl)-ethyl]-cyclohexyl)-3-(1H-indol-3-ylmethyl)urea;
1-(trans-4-[2-(1,2,3,4-tetrahydro-1,4-epiazano-naphthalen-9-yl)-ethyl]-cyclohexyl)-3-(1H-benzimidazol-2-ylmethyl)urea;
1-(trans-4-[2-(1,2,3,4-tetrahydro-1,4-epiazano-naphthalen-9-yl)-ethyl]-cyclohexyl)-3-(1,2,3,4-tetrahydro-2-naphthalenyl)urea;
1-(trans-4-[2-(1,2,3,4-tetrahydro-1,4-epiazano-naphthalen-9-yl)-ethyl]-cyclohexyl)-3-(1,2,3,4-tetrahydro-1-naphthalenyl)urea;
1-(trans-4-[2-(1,2,3,4-tetrahydro-1,4-epiazano-naphthalen-9-yl)-ethyl]-cyclohexyl)-N,N-dimethylphenylalaninamide;
1-(trans-4-[2-(1,2,3,4-tetrahydro-1,4-epiazano-naphthalen-9-yl)-ethyl]-cyclohexyl)-3-(4-phenylbutyl)urea;
1-(trans-4-[2-(1,2,3,4-tetrahydro-1,4-epiazano-naphthalen-9-yl)-ethyl]-cyclohexyl)-3-(2-methyl-1,2,3,4-tetrahydro-2-naphthalenyl)urea;
1-(trans-4-[2-(1,2,3,4-tetrahydro-1,4-epiazano-naphthalen-9-yl)-ethyl]-cyclohexyl)-3-(2-methyl-1,2,3,4-tetrahydro-2-naphthalenyl)urea;
N-(trans-4-[2-(1,2,3,4-tetrahydro-1,4-epiazano-naphthalen-9-yl)-ethyl]-cyclohexyl)-4-(2-pyridinyl)-1-piperazinecarboxamide;
1-(trans-4-[2-(1,2,3,4-tetrahydro-1,4-epiazano-naphthalen-9-yl)-ethyl]-cyclohexyl)-3-[2-(4-pyridinyl)ethyl]urea formate;
N-(trans-4-[2-(1,2,3,4-tetrahydro-1,4-epiazano-naphthalen-9-yl)-ethyl]-cyclohexyl)-2,2-diphenylacetamide;
N-(trans-4-[2-(1,2,3,4-tetrahydro-1,4-epiazano-naphthalen-9-yl)-ethyl]-cyclohexyl)-2,2-diphenylacetamide;
1-(trans-4-[2-(1,2,3,4-tetrahydro-1,4-epiazano-naphthalen-9-yl)-ethyl]-cyclohexyl)-3-[3-(1H-imidazol-1-yl)propyl]urea formate;
1-(trans-4-[2-(1,2,3,4-tetrahydro-1,4-epiazano-naphthalen-9-yl)-ethyl]-cyclohexyl)-3-{[4-(trifluoromethyl)phenyl]methyl}urea;
N-(trans-4-[2-(1,2,3,4-tetrahydro-1,4-epiazano-naphthalen-9-yl)-ethyl]-cyclohexyl)-4-(phenylmethyl)-1-piperazinecarboxamide;
N-{5-[(trans-4-[2-(1,2,3,4-tetrahydro-1,4-epiazano-naphthalen-9-yl)-ethyl]-cyclohexyl)amino]-5-oxopentyl)benzamide;
1-(trans-4-[2-(1,2,3,4-tetrahydro-1,4-epiazano-naphthalen-9-yl)-ethyl]-cyclohexyl)-3-(1H-indol-3-ylmethyl)urea formate;
1-(trans-4-[2-(1,2,3,4-tetrahydro-1,4-epiazano-naphthalen-9-yl)-ethyl]-cyclohexyl)-3-{[3-(dimethylamino)phenyl]methyl}urea formate;
N-(trans-4-[2-(1,2,3,4-tetrahydro-1,4-epiazano-naphthalen-9-yl)-ethyl]-cyclohexyl)-3-(4-methylphenyl)-3-phenylpropanamide;
N-(trans-4-[2-(1,2,3,4-tetrahydro-1,4-epiazano-naphthalen-9-yl)-ethyl]-cyclohexyl)-4,4-diphenylbutanamide;
1-(trans-4-[2-(1,2,3,4-tetrahydro-1,4-epiazano-naphthalen-9-yl)-ethyl]-cyclohexyl)-2-(methyloxy)-2,2-diphenylacetamide;
1-(trans-4-[2-(1,2,3,4-tetrahydro-1,4-epiazano-naphthalen-9-yl)-ethyl]-cyclohexyl)-3-(1-naphthalenylmethyl)urea formate;
N-(trans-4-[2-(1,2,3,4-tetrahydro-1,4-epiazano-naphthalen-9-yl)-ethyl]-cyclohexyl)-4-(phenylmethyl)-1-piperazinecarboxamide formate;
1-(trans-4-[2-(1,2,3,4-tetrahydro-1,4-epiazano-naphthalen-9-yl)-ethyl]-cyclohexyl)-3-(2-{3-[hydroxy(3-pyridinyl)methyl]phenyl}ethyl)urea;
1-(trans-4-[2-(1,2,3,4-tetrahydro-1,4-epiazano-naphthalen-9-yl)-ethyl]-cyclohexyl)-3-[1-(phenylmethyl)-4-piperidinyl]urea formate;
1-(trans-4-[2-(1,2,3,4-tetrahydro-1,4-epiazano-naphthalen-9-yl)-ethyl]-cyclohexyl)-3-(3-phenylpropyl)urea trifluoroacetate;
1-(trans-4-[2-(1,2,3,4-tetrahydro-1,4-epiazano-naphthalen-9-yl)-ethyl]-cyclohexyl)-3-[5,8-bis(methyloxy)-1,2,3,4-tetrahydro-2-naphthalenyl]urea formate;
N′-(trans-4-[2-(1,2,3,4-tetrahydro-1,4-epiazano-naphthalen-9-yl)-ethyl]-cyclohexyl)-N-(3,3-diphenylpropyl)-N-propylurea;
1-(trans-4-[2-(1,2,3,4-tetrahydro-1,4-epiazano-naphthalen-9-yl)-ethyl]-cyclohexyl)-3-(3,3-diphenylpropyl)urea formate;
1-(trans-4-[2-(1,2,3,4-tetrahydro-1,4-epiazano-naphthalen-9-yl)-ethyl]-cyclohexyl)-3-(1-methyl-2,2-diphenylethyl)urea formate;
N-(trans-4-[2-(1,2,3,4-tetrahydro-1,4-epiazano-naphthalen-9-yl)-ethyl]-cyclohexyl)-2-[(2-methylphenyl)(phenyl)methyl]benzamide;
N-(trans-4-[2-(1,2,3,4-tetrahydro-1,4-epiazano-naphthalen-9-yl)-ethyl]-cyclohexyl)-4-(diethylamino)-2,2-diphenylbutanamide;
1-(trans-4-[2-(1,2,3,4-tetrahydro-1,4-epiazano-naphthalen-9-yl)-ethyl]-cyclohexyl)-3-(2-{3-[hydroxy(3-pyridinyl)methyl]phenyl}ethyl)urea formate;
1-(trans-4-[2-(1,2,3,4-tetrahydro-1,4-epiazano-naphthalen-9-yl)-ethyl]-cyclohexyl)-3-(1,1-dimethyl-3,3-diphenylpropyl)urea formate;
N′-(trans-4-[2-(1,2,3,4-tetrahydro-1,4-epiazano-naphthalen-9-yl)-ethyl]-cyclohexyl)-N-(3,3-diphenylpropyl)-N-ethylurea formate;
N′-(trans-4-[2-(1,2,3,4-tetrahydro-1,4-epiazano-naphthalen-9-yl)-ethyl]-cyclohexyl)-N-methyl-N-(2,2,2-triphenylethyl)urea;
N-(trans-4-[2-(1,2,3,4-tetrahydro-1,4-epiazano-naphthalen-9-yl)-ethyl]-cyclohexyl)-3-phenyl-3-{3-[(phenylmethyl)oxy]phenyl}propanamide;
N-(trans-4-[2-(1,2,3,4-tetrahydro-1,4-epiazano-naphthalen-9-yl)-ethyl]-cyclohexyl)-2-hydroxy-2,2-diphenylacetamide trifluoroacetate;
N′-(trans-4-[2-(1,2,3,4-tetrahydro-1,4-epiazano-naphthalen-9-yl)-ethyl]-cyclohexyl)-N-ethyl-N-(3-hydroxy-3,3-diphenylpropyl)urea formate;
N-(trans-4-[2-(1,2,3,4-tetrahydro-1,4-epiazano-naphthalen-9-yl)-ethyl]-cyclohexyl)-2-{bis[4-(dimethylamino)phenyl]methyl}benzamide;
N-(trans-4-[2-(1,2,3,4-tetrahydro-1,4-epiazano-naphthalen-9-yl)-ethyl]-cyclohexyl)-3-[4-(dimethylamino)phenyl]-3-phenylpropanamide trifluoroacetate;
N′-(trans-4-[2-(1,2,3,4-tetrahydro-1,4-epiazano-naphthalen-9-yl)-ethyl]-cyclohexyl)-N-(3,3-diphenylpropyl)-N-(phenylmethyl)urea formate;
N-(trans-4-[2-(1,2,3,4-tetrahydro-1,4-epiazano-naphthalen-9-yl)-ethyl]-cyclohexyl)-2,2-bis(4-chlorophenyl)acetamide trifluoroacetate;
N-(trans-4-[2-(1,2,3,4-tetrahydro-1,4-epiazano-naphthalen-9-yl)-ethyl]-cyclohexyl)-4-(diethylamino)-2,2-diphenylbutanamide trifluoroacetate;
1-(trans-4-[2-(1,2,3,4-tetrahydro-1,4-epiazano-naphthalen-9-yl)-ethyl]-cyclohexyl)-3-[3-(4-biphenylyl)-3-(4-chlorophenyl)-3-hydroxypropyl]urea formate;
1-(4-Bromo-benzyl)-3-(4-[2-(1,2,3,4-tetrahydro-1,4-epiazano-naphthalen-9-yl)-ethyl]-cyclohexyl}-urea;
1-(1,1-Diphenyl-methyl)-3-{4-[2-(1,2,3,4-tetrahydro-1,4-epiazano-naphthalen-9-yl)-ethyl]-cyclohexyl}-urea;
1-(2-Methoxy-benzyl)-3-{4-[2-(1,2,3,4-tetrahydro-1,4-epiazano-naphthalen-9-yl)-ethyl]-cyclohexyl}-urea;
1-(3-Methoxy-benzyl)-3-{4-[2-(1,2,3,4-tetrahydro-1,4-epiazano-naphthalen-9-yl)-ethyl]-cyclohexyl}-urea;
1-(4-Methoxy-benzyl)-3-{4-[2-(1,2,3,4-tetrahydro-1,4-epiazano-naphthalen-9-yl)-ethyl]-cyclohexyl}-urea;
2-Methyl-quinoline-5-carboxylic acid {4-[2-(1,4-dihydro-1,4-epiazano-naphthalen-9-yl)-ethyl]-cyclohexyl}-amide;
8-Chloro-2-methyl-quinoline-5-carboxylic acid {4-[2-(1,4-dihydro-1,4-epiazano-naphthalen-9-yl)-ethyl]-cyclohexyl}-amide;
8-Methoxy-2-methyl-quinoline-5-carboxylic acid {4-[2-(1,4-dihydro-1,4-epiazano-naphthalen-9-yl)-ethyl]-cyclohexyl}-amide;
Quinoxaline-5-carboxylic acid {4-[2-(1,4-dihydro-1,4-epiazano-naphthalen-9-yl)-ethyl]-cyclohexyl}-amide;
Quinoline-5-carboxylic acid {trans-4-[1-(1,2,3,4-tetrahydro-1,4-epiazano-naphthalen-9-yl)methyl]-cyclohexylmethyl}-amide;
8-Methyl-quinoline-5-carboxylic acid {trans-4-[1-(1,2,3,4-tetrahydro-1,4-epiazano-naphthalen-9-yl)methyl]-cyclohexylmethyl}-amide;
2-Methyl-quinoline-5-carboxylic acid {trans-4-[(1S,4S)-1-(1,2,3,4-tetrahydro-1,4-epiazano-naphthalen-9-yl) methyl]-cyclohexylmethyl}-amide;
8-Chloro-2-methyl-quinoline-5-carboxylic acid {trans-4-[1-(1,2,3,4-tetrahydro-1,4-epiazano-naphthalen-9-yl)methyl]-cyclohexylmethyl}-amide;
2,8-Dimethyl-quinoline-5-carboxylic acid {trans-4-[1-(1,2,3,4-tetrahydro-1,4-epiazano-naphthalen-9-yl)methyl]-cyclohexylmethyl}-amide;
1-((S)-1-Naphthalen-1-yl-ethyl)-3-{trans-4-[1-(1,2,3,4-tetrahydro-1,4-epiazano-naphthalen-9-yl)methyl]-cyclohexylmethyl}-urea;
1-((R)-1-Naphthalen-1-yl-ethyl)-3-{trans-4-[(1-(1,2,3,4-tetrahydro-1,4-epiazano-naphthalen-9-yl)methyl]-cyclohexylmethyl}-urea;
Isoquinoline-1-carboxylic acid {trans-4-[(1S,4R)-2-(1,2,3,4-tetrahydro-1,4-epiazano-naphthalen-9-yl)-ethyl]-cyclohexyl}-amide;
Acridine-9-carboxylic acid {trans-4-[2-(1,2,3,4-tetrahydro-1,4-epiazano-naphthalen-9-yl)-ethyl]-cyclohexyl}-amide;
2,3-Dihydro-naphthalene-1-carboxylic acid {trans-4-[2-(1,2,3,4-tetrahydro-1,4-epiazano-naphthalen-9-yl)-ethyl]-cyclohexyl}-amide;
6,7-Dihydro-quinoline-8-carboxylic acid {trans-4-[2-(1,2,3,4-tetrahydro-1,4-epiazano-naphthalen-9-yl)-ethyl]-cyclohexyl}-amide;
9-[2-(trans-4-{[1-(2-Methyl-quinolin-5-yl)-methanoyl]-amino}-cyclohexyl)-ethyl]-1,4-dihydro-1,4-epiazano-naphthalene-6-carboxylic acid methyl ester;
9-(2-{trans-4-[3-((S)-1-Naphthalen-1-yl-ethyl)-ureido]-cyclohexyl}-ethyl)-1,4-dihydro-1,4-epiazano-naphthalene-6-carboxylic acid methyl ester;
9-[2-(trans-4-{[1-(2-Methyl-quinolin-5-yl)-methanoyl]-amino}-cyclohexyl)-ethyl]-1,2,3,4-tetrahydro-1,4-epiazano-naphthalene-6-carboxylic acid methyl ester;
(1S,4R)-9-(2-{4-[(1-Quinolin-5-yl-methanoyl)-amino]-cyclohexyl}-ethyl)-1,2,3,4-tetrahydro-1,4-epiazano-naphthalene-6-carboxylic acid methyl ester;
9-(2-{trans-4-[3-((S)-1-Naphthalen-1-yl-ethyl)-ureido]-cyclohexyl}-ethyl)-1,2,3,4-tetrahydro-1,4-epiazano-naphthalene-6-carboxylic acid methyl ester;
1-(trans-4-{2-[6-(2-Methyl-propanoyl)-1,2,3,4-tetrahydro-1,4-epiazano-naphthalen-9-yl]-ethyl}-cyclohexyl)-3-((S)-1-naphthalen-1-yl-ethyl)-urea;
Quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
8-Chloro-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
8-Chloro-2-methyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
8-Methyl-quinoline-5-carboxylic acid (4-{2-[(1S,4R)-6-(2-methyl-propanoyl)-1,2,3,4-tetrahydro-1,4-epiazano-naphthalen-9-yl]-ethyl}-cyclohexyl)-amide;
2,8-Dimethyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
1-(trans-4-{2-[6-(2-Methyl-propanoyl)-1,2,3,4-tetrahydro-1,4-epiazano-naphthalen-9-yl]-ethyl}-cyclohexyl)-3-((R)-1-naphthalen-1-yl-ethyl)-urea;
1-(trans-4-{2-[6-Butyryl-1,2,3,4-tetrahydro-1,4-epiazano-naphthalen-9-yl]-ethyl}-cyclohexyl)-3-((S)-1-naphthalen-1-yl-ethyl)-urea;
Quinoline-5-carboxylic acid (trans-4-{2-[6-butyryl-propanoyl)-1,2,3,4-tetrahydro-1,4-epiazano-naphthalen-9-yl]-ethyl}-cyclohexyl)-amide;
8-Chloro-2-methyl-quinoline-5-carboxylic acid (trans-4-{2-[6-butyryl-1,2,3,4-tetrahydro-1,4-epiazano-naphthalen-9-yl]-ethyl}-cyclohexyl)-amide;
Quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
1-((S)-1-Naphthalen-1-yl-ethyl)-3-(trans-4-{2-[6-(2-phenyl-ethanoyl)-1,2,3,4-tetrahydro-1,4-epiazano-naphthalen-9-yl]-ethyl}-cyclohexyl)-urea;
8-Methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
2,8-Dimethyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
8-Methoxy-2-methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
1-((R)-1-Naphthalen-1-yl-ethyl)-3-(4-{2-[(1S,4R)-6-(2-phenyl-ethanoyl)-1,2,3,4-tetrahydro-1,4-epiazano-naphthalen-9-yl]-ethyl}-cyclohexyl)-urea;
Quinoline-5-carboxylic acid methyl-{trans-4-[1-(1,2,3,4-tetrahydro-1,4-epiazano-naphthalen-9-yl)methyl]-cyclohexylmethyl}-amide 8-Methyl-quinoline-5-carboxylic acid methyl-{trans-4-[1-(1,2,3,4-tetrahydro-1,4-epiazano-naphthalen-9-yl)methyl]-cyclohexylmethyl}-amide;
2,8-Dimethyl-quinoline-5-carboxylic acid methyl-{trans-4-[1-(1,2,3,4-tetrahydro-1,4-epiazano-naphthalen-9-yl)methyl]-cyclohexylmethyl}-amide;
8-Chloro-quinoline-5-carboxylic acid methyl-{4-[(1S,4S)-1-(1,2,3,4-tetrahydro-1,4-epiazano-naphthalen-9-yl)methyl]-cyclohexylmethyl}-amide;
8-Chloro-2-methyl-quinoline-5-carboxylic acid methyl-{trans-4-[1-(1,2,3,4-tetrahydro-1,4-epiazano-naphthalen-9-yl)methyl]-cyclohexylmethyl}-amide and pharmaceutically acceptable salts thereof.

### Methods of Preparation

The compounds of Formula (I) may be obtained by applying synthetic procedures, some of which are illustrated in the Schemes below. The synthesis provided for these Schemes is applicable for producing compounds of Formula (I) having a variety of different R¹, R², G¹ and G² which are reacted, employing substituents which are suitable protected, to achieve compatibility with the reactions outlined herein. Subsequent deprotection, in those cases, then affords compounds of the nature generally disclosed. Once the bicyclic amine core has been established, further compounds of these Formulas may be prepared by applying techniques for functional groups interconversion, well known in the art. While the Schemes are shown with compounds only of Formula (i), this is merely for illustration purpose only. Scheme 1

The desired compounds of formula (I) can be prepared as outlined in Scheme 1. Compounds 3 can be obtained via a benzyne reaction from suitable starting materials such as 2-fluorobromobenzenes and suitably N-protected pyrrole using carbamate protecting groups well known in the art such as the Boc group. The reaction can be effected using reagents such as magnesium or alkyl lithiums in suitable solvent such as THF or ether. Compounds 5 can be obtained by deprotection of the Boc group using standard methods such as treatment with trifluroacetic acid (TFA), dry HCl or iodotrimethylsilane (TMSI) in suitable aprotic solvents. The compounds 4 can be prepared by subjecting 3 to standard reductive conditions well known to those skilled in the art such as treatment with hydrogen gas in the presence of a catalytic amount of palladium on carbon in a suitable solvent such as ethanol. Deproctection to yield compounds 6 can be effected in a manner similar to that described for compounds 5. Compounds 8 can be obtained by reacting 5 or 6 with aldehydes 7 under the well known reductive amination conditions using suitable reagents such as sodium triacetoxyborohydride. The 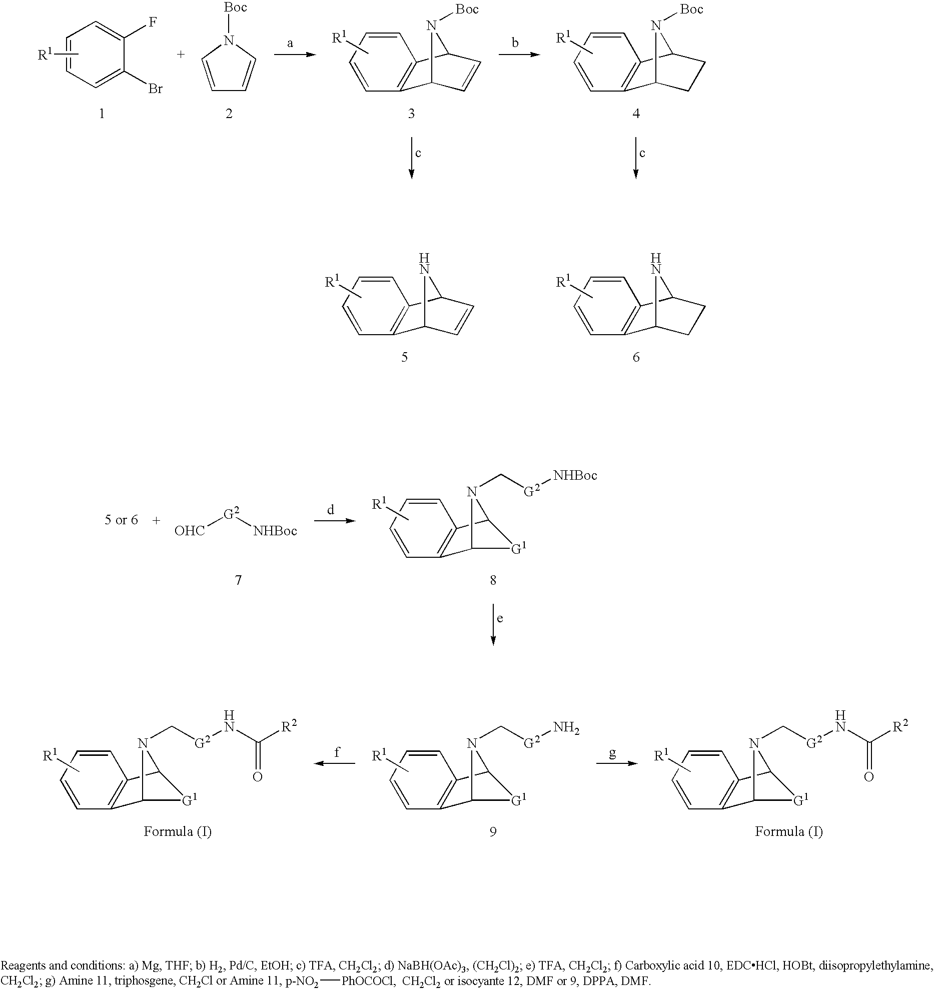
compounds 9 can then be prepared by deprotection of 8 using the conditions listed for the preparation of the compounds 5. Compounds of formula (I) which are of the amide type, can be made by treating compounds 9 with carboxylic acids 10 under suitable amide coupling conditions well known to those skilled in the art such as 1-hydroxybenzotriazole hydrate (HOBt), 1-(3-dimethylaminopropyl)-3-ethylcarbodiimide hydrochloride (EDC HCl) and diisopropylethylamine(DIEA) in dichloromethane. Compounds of formula (I) which are of the urea type, can be made by treating compounds 9 with a suitable coupling reagent such as triphosgene or 4-nitrophenylchloroformate followed by amines 11 or by treating compounds 9 with isocyanates 12, which may have been formed in situ via a Curtius rearrangement effected by exposing carboxylic acids 10 a reagent such as diphenylphosphoryl azide, in a suitable solvent such as DMF.

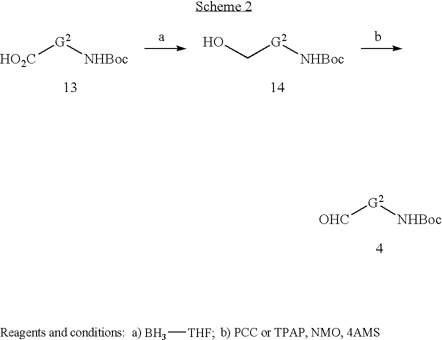

Aldehydes 4 may be prepared from carboxylic acids 13 by reduction to the alcohol 14 using standard conditions such as borane-THF complex (BH₃-THF) followed by oxidation to the aldehyde using standard conditions well know to those skilled in the art such as pyridinium chlorochromate (PCC), tetrapropylammonium perruthenate (TPAP), Swern oxidation or Dess-Martin periodinane. Alternatively, compounds 4 may be prepared
according to Stemp et al. (J. Med. Chem. 2000, 43, 1878-85).

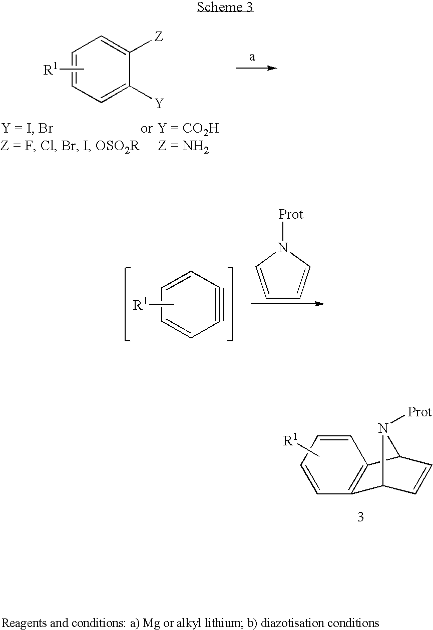

If suitable 2-fluorobromobenzenes are not commercially available, the benzyne reaction to form compounds 3 can be performed with other 1,2-substituted benzenes: 1) For those in which the substituent Y is either iodine or bromine and the substituent Z is any halogen or an aryl sulfonate the benzyne forming reaction may be effected by treatment with either magnesium or an alkyl lithium; 2) For 2-aminobenzoic acids, the benzyne may be formed by subjecting the substrate to diazotisation reagents well know in the art such as isoamylnitrite or sodium nitrite in acidic media.

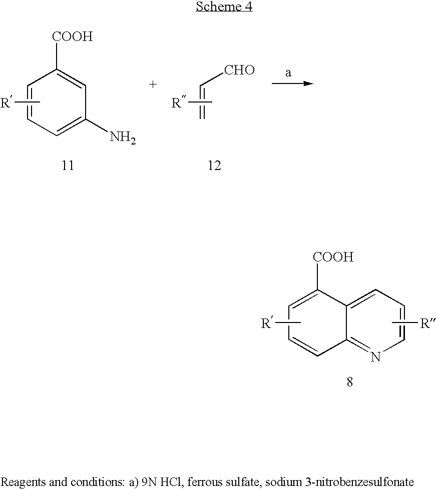

If the required acid 8 is of the quinoline-5-carboxylic acid-type, it can be prepared as outlined in Scheme 3. The 3-amino-benzolic acid 11 can be converted to quinoline-5-carboxylic acid 8 by condensing with a suitable propenal 12. Otherwise, non-commercially available acids 8 can be prepared as described by Hadley et al. (WO 00/21951).

A more specific preparation method leading to compounds with Formula (I) is outlined in Scheme 5. 1,1′-Carbonyldiimidazole (CDI) mediated condensation of amine 15 with acid 16 provided amide 17. Reduction with lithium aluminium hydride (LAH) afforded amine 18 that was coupled with acid 19 under suitable amide formation conditions well known to those skilled in the art such as EDC and HOBt to generate compound 20.

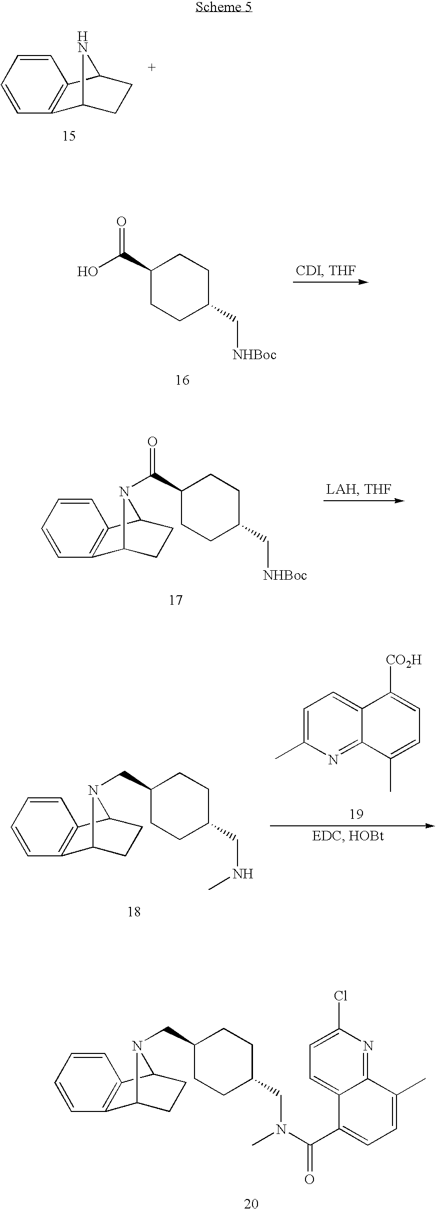

A more specific preparation method leading to compounds with Formula (I) is outlined in Scheme 6. Starting with benzyne formation from 21, coupling with pyrrole 22 provided 23. Reduction of the olefine with H₂ followed by reduction of the ester with super hydride and oxidation of the resultant alcohol with MnO₂ then afforded aldehyde 26. Addition of CH₃CH₂CH₂MgBr followed by oxidation with MnO₂ and deprotection with TFA furnished bicyclic amine 29. Condensation with aldehyde 30, reduction of the resultant imine with NaBH(OAc)₃ and deprotection with TFA generated primary amine 32. It was conveniently converted to amide 34 or urea 36.

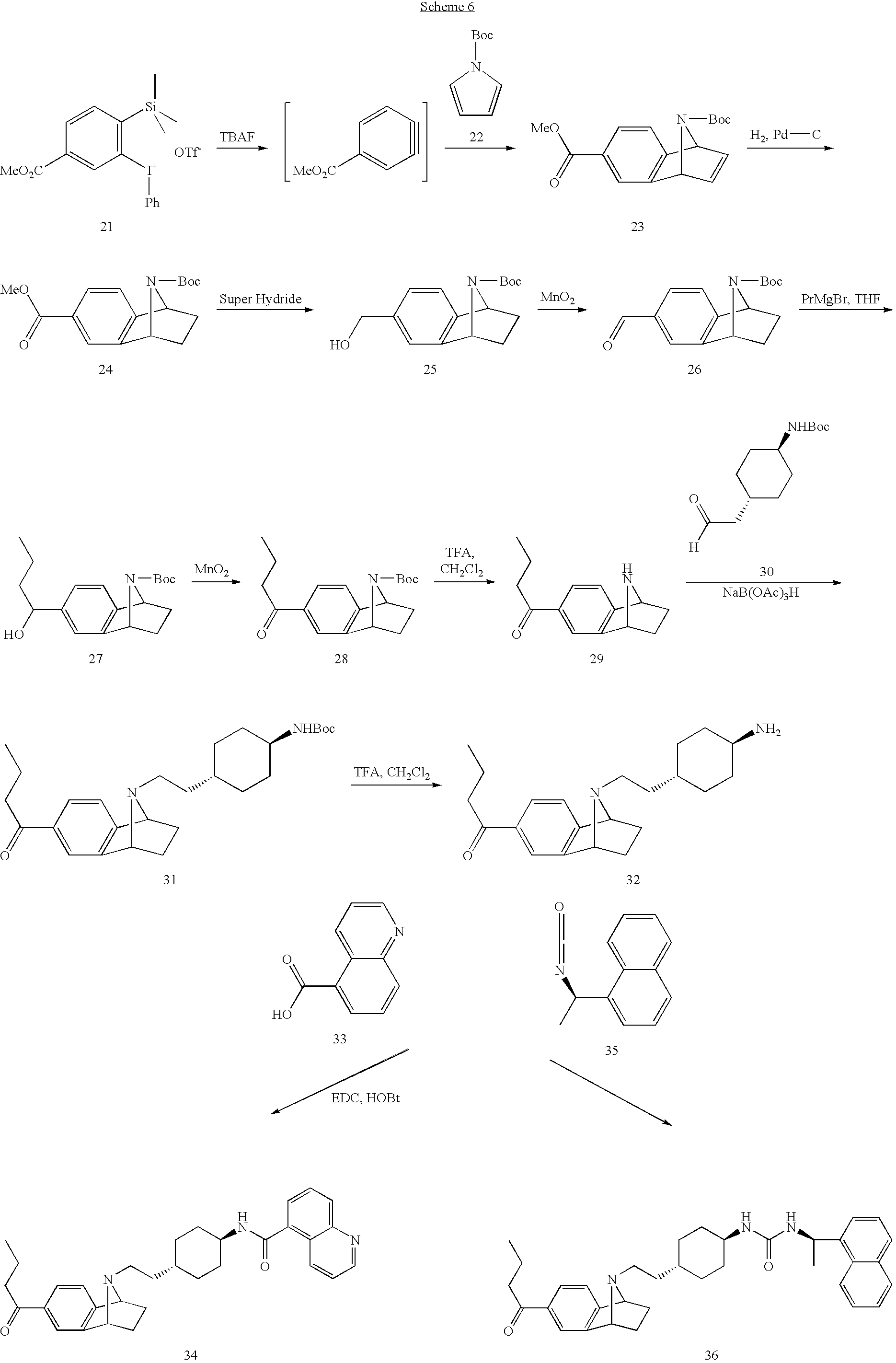

### SYNTHETIC EXAMPLES

### Example 1

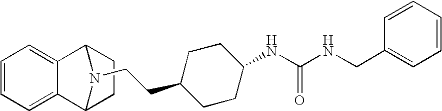

### 1-Benzyl-3-{4-[2-(1,2,3,4-tetrahydro-1,4-epiazano-naphthalen-9-yl)-ethyl]-cyclohexyl}-urea

### 1a) {4-[2-(1,2,3,4-Tetrahydro-1,4-epiazano-naphthalen-9-yl)-ethyl]-cyclohexyl}-carbamic acid tert-butyl ester

To a solution of 1,2,3,4-tetrahydro-1,4-epiazano-naphthalene (prepared to J. of Organic Chemistry, 1966, 31, 764-767) (1.237 g, 8.52 mmol) in 75 mL of 1,2-dichloroethane, [4-(2-oxo-ethyl)-cyclohexyl]-carbamic acid tert-butyl ester (2.056 g, 8.52 mmol) and sodium triacetoxyborohydride (2.708 g, 12.78 mmol) were added. The mixture was stirred at RT overnight, diluted with dichloromethane. The resulting mixture was washed with saturated aqueous K₂CO₃, extracted with dichloromethane, dried over magnesium sulfate and removed the solvent in vacuo. The resulting crude was purified via filtering through a silica pad using ethyl acetate as mobile phase to give 2.681 g (85%) of the title compound. LCMS m/z 371.2 (M+H).

### 1b) 4-[2-(1,2,3,4-Tetrahydro-1,4-epiazano-naphthalen-9-yl)-ethyl]-cyclohexylamine

To the solution of the crude {4-[2-(1,2,3,4-tetrahydro-1,4-epiazano-naphthalen-9-yl)-ethyl]-cyclohexyl}-carbamic acid tert-butyl ester (2.681 g, 7.23 mmol) in 60 mL of dichloromethane was added 4.5 mL of TFA. The mixture was stirred at RT overnight. The solvent was evaporated to give the crude TFA salt. This material was partitioned between saturated aqueous K₂CO₃ and CH₂Cl₂. The mixture was extracted with CH₂Cl₂ (2×). The combined organic phase was washed with brine, dried over magnesium sulfate and removed the solvent in vacuo to give the title compound 1.759 g (90%). LCMS m/z 271.2 (M+H).

### 1c) 1-Benzyl-3-{4-[2-(1,2,3,4-tetrahydro-1,4-epiazano-naphthalen-9-yl)-ethyl]-cyclohexyl}-urea

The mixture of 4-[2-(1,2,3,4-tetrahydro-1,4-epiazano-naphthalen-9-yl)-ethyl]-cyclohexylamine (137 mg, 0.51 mmol) and benzyl isocyanate (63 μL, 0.51 mmol) in 1.4 mL of DMF was stirred at RT overnight. Solid precipitated out of the solution. The mixture was filtered, the solid was washed with ethyl acetate and hexane to yield the title compound 131 mg (65%) as a white solid. LCMS m/z 404.2 (M+H).

### Example 2

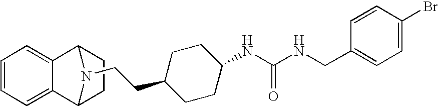

### 1-(4-Bromo-benzyl)-3-{4-[2-(1,2,3,4-tetrahydro-1,4-epiazano-naphthalen-9-yl)-ethyl]-cyclohexyl}-urea

Following the general procedure described in Example 1c, 4-[2-(1,2,3,4-tetrahydro-1,4-epiazano-naphthalen-9-yl)-ethyl]-cyclohexylamine (76 mg, 0.28 mmol) coupled with 4-bromo-benzyl isocyanate (40 μL, 0.28 mmol) to give the titled compound 37 mg (28%). LCMS m/z 482.0 (M+H).

### Example 3

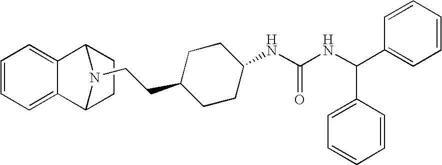

### 1-(1,1-Diphenyl-methyl)-3-{4-[2-(1,2,3,4-tetrahydro-1,4-epiazano-naphthalen-9-yl)-ethyl]-cyclohexyl}-urea

The mixture of 4-[2-(1,2,3,4-tetrahydro-1,4-epiazano-naphthalen-9-yl)-ethyl]-cyclohexylamine (68 mg, 0.25 mmol) and diphenylmethyl isocyanate (48 μL, 0.25 mmol) in 1.0 mL of DMF was stirred at RT overnight. The mixture was diluted with ethyl acetate, washed with water (2×) and dried over magnesium sulfate. Removal of the solvent in vacuo and recrystallization from ethyl acetate/hexane gave the titled compound 55 mg (46%). LCMS m/z 480.2 (M+H).

### Example 4

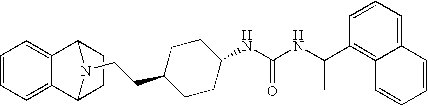

### 1-(1-Naphthalen-1-yl-ethyl)-3-{4-[2-(1,2,3,4-tetrahydro-1,4-epiazano-naphthalen-9-yl)-ethyl]-cyclohexyl}-urea

Following the general procedure described in Example 3, 4-[2-(1,2,3,4-tetrahydro-1,4-epiazano-naphthalen-9-yl)-ethyl]-cyclohexylamine (75 mg, 0.28 mmol) coupled with 1-(1-naphthyl)ethyl isocyanate (49 μL, 0.28 mmol) to give the titled compound 62 mg (52%). LCMS m/z 468.4 (M+H).

### Example 5

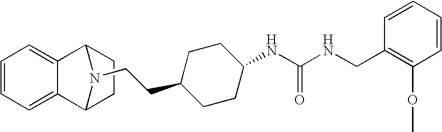

### 1-(2-Methoxy-benzyl)-3-{4-[2-(1,2,3,4-tetrahydro-1,4-epiazano-naphthalen-9-yl)-ethyl]-cyclohexyl}-urea

Following the general procedure described in Example 1c, 4-[2-(1,2,3,4-tetrahydro-1,4-epiazano-naphthalen-9-yl)-ethyl]-cyclohexylamine (66 mg, 0.24 mmol) coupled with 2-methoxy-benzyl isocyanate (38 μL, 0.24 mmol) to give the titled compound 72 mg (72%). LCMS m/z 434.4 (M+H).

### Example 6

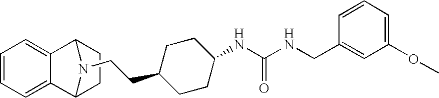

### 1-(3-Methoxy-benzyl)-3-{4-[2-(1,2,3,4-tetrahydro-1,4-epiazano-naphthalen-9-yl)-ethyl]-cyclohexyl}-urea

Following the general procedure described in Example 1c, 4-[2-(1,2,3,4-tetrahydro-1,4-epiazano-naphthalen-9-yl)-ethyl]-cyclohexylamine (67 mg, 0.25 mmol) coupled with 3-methoxy-benzyl isocyanate (36 μL, 0.25 mmol) to give the titled compound 31 mg (31%). LCMS m/z 434.4 (M+H).

### Example 7

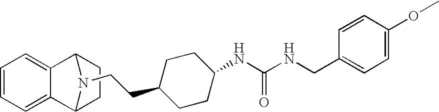

### 1-(4-Methoxy-benzyl)-3-{4-[2-(1,2,3,4-tetrahydro-1,4-epiazano-naphthalen-9-yl)-ethyl]-cyclohexyl}-urea

Following the general procedure described in Example 1c, 4-[2-(1,2,3,4-tetrahydro-1,4-epiazano-naphthalen-9-yl)-ethyl]-cyclohexylamine (77 mg, 0.28 mmol) coupled with 4-methoxy-benzyl isocyanate (41 μL, 0.28 mmol) to give the titled compound 49 mg (%). LCMS m/z 434.4 (M+H).

### Example 8

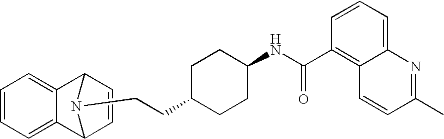

### 2-Methyl-quinoline-5-carboxylic acid {4-[2-(1,4-dihydro-1,4-epiazano-naphthalen-9-yl)-ethyl]-cyclohexyl}-amide

### 8a) {4-[2-(1,4-Dihydro-1,4-epiazano-naphthalen-9-yl)-ethyl]-cyclohexyl}-carbamic acid tert-butyl ester

Following the procedure outlined in Example 1a, 1,4-dihydro-1,4-epiazano-naphthalene, which was made according to J. of Organic Chemistry, 1966, 31, 764-767, (825 mg, 5.73 mmol) was treated with [4-(2-oxo-ethyl)-cyclohexyl]-carbamic acid tert-butyl ester (1.38 g, 5.73 mmol) in the presence of sodium triacetoxyborohydride (1.81 g, 8.57 mmol) to afford the crude material. Flash chromatography on silica gel eluting with methanol/dichloromethane (5/95, v/v) to give 1.52 g (72%) of the title compound. LCMS m/z 369 (M+H).

### 8b) 4-[2-(1,4-Dihydro-1,4-epiazano-naphthalen-9-yl)-ethyl]-cyclohexylamine

The mixture of {4-[2-(1,4-dihydro-1,4-epiazano-naphthalen-9-yl)-ethyl]-cyclohexyl}-carbamic acid tert-butyl ester (760 mg, 2.14 mmol) and iodotrimethylsilane (455 μL, 3.2 mmol) in 10 mL of chloroform was stirred at RT for 2 h. Removal the solvent in vacuo yielded the crude material 0.89 g, which was used in the next step without further purification.

### 8c) 2-Methyl-quinoline-5-carboxylic acid {4-[2-(1,4-dihydro-1,4-epiazano-naphthalen-9-yl)-ethyl]-cyclohexyl}-amide

To a mixture of 4-[2-(1,4-dihydro-1,4-epiazano-naphthalen-9-yl)-ethyl]-cyclohexylamine (150 mg, 0.59 mmol), DIEA (514 μL, 2.95 mmol) and 2-methyl-quinoline-5-carboxylic 5 acid (145 mg, 0.65 mmol) in 15 mL of chloroform were added EDC (113 mg, 0.55 mmol) and HOBT (8 mg, 0.059 mmol). The mixture was stirred at RT overnight. The solvent was removed in vacuo to yield the crude product. Purification upon Gilson HPLC, eluting with acetonitrile/water/0.1% TFA (10/90, v/v to 70/30, v/v, over 10 min), gave the desired product 232 mg (90%). LCMS: m/z 438 (M+H).

### Example 9

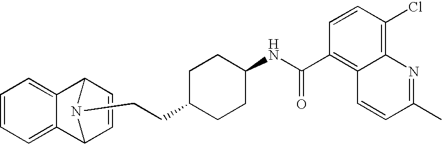

### 8-Chloro-2-methyl-quinoline-5-carboxylic acid {-4-[2-(1,4-dihydro-1,4-epiazano-naphthalen-9-yl)-ethyl]-cyclohexyl}-amide

Following the general procedure outlined in Example 8c, 4-[2-(1,4-dihydro-1,4-epiazano-naphthalen-9-yl)-ethyl]-cyclohexylamine (150 mg, 0.59 mmol) coupled with 8-chloro-2-methyl-quinoline-5-carboxylic acid (167 mg, 0.65 mmol) to afford the title compound 18 mg. LCMS 472 (M+H).

### Example 10

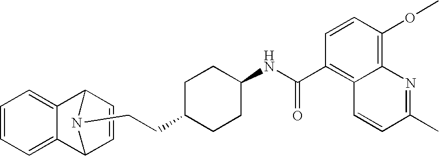

### 8-Methoxy-2-methyl-quinoline-5-carboxylic acid {4-[2-(1,4-dihydro-1,4-epiazano-naphthalen-9-yl)-ethyl]-cyclohexyl}-amide

Following the general procedure outlined in Example 8c, 4-[2-(1,4-dihydro-1,4-epiazano-naphthalen-9-yl)-ethyl]-cyclohexylamine (150 mg, 0.59 mmol) coupled with 8-methoxy-2-methyl-quinoline-5-carboxylic acid (165 mg, 0.64 mmol) to afford the title compound 266 mg (96%). LCMS 468 (M+H).

### Example 11

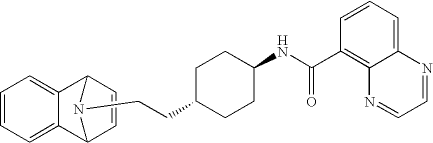

### Quinoxaline-5-carboxylic acid {4-[2-(1,4-dihydro-1,4-epiazano-naphthalen-9-yl)-ethyl]-cyclohexyl}-amide

Following the general procedure outlined in Example 8c, 4-[2-(1,4-dihydro-1,4-epiazano-naphthalen-9-yl)-ethyl]-cyclohexylamine (100 mg, 0.37 mmol) coupled with quinoxaline-5-carboxylic acid (71 mg, 0.41 mmol) to afford the title compound 117 mg (74%). LCMS 425 (M+H).

### Example 12

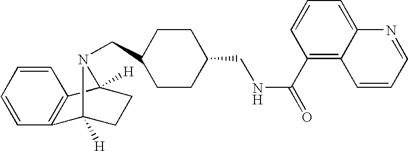

### Quinoline-5-carboxylic acid {trans-4-[1-(1,2,3,4-tetrahydro-1,4-epiazano-naphthalen-9-yl)methyl]-cyclohexylmethyl}-amide

### 12a) Preparation of [trans-4-(1,2,3,4-tetrahydro-1,4-epiazano-naphthalen-9-yl-methanoyl)-cyclohexylmethyl]-carbamic acid tert-butyl ester

To a solution of trans-4-[({[(1,1-dimethylethyl)oxy]carbonyl}amino)methyl]cyclohexanecarboxylic acid (1.9 g, 7.6 mmol) in THF (10 mL), 1,1′-carbonyldiimidazole (1.2 mL, 7.6 mmol) was added. The mixture was stirred at room temperature for 30 minutes before 1,2,3,4-tetrahydro-1,4-epiazano-naphthalene (1.0 g, 6.9 mmol) was added. The resultant solution was stirred at room temperature for 3 hours, diluted with EtOAc (50 mL), and washed with H₂O (30 mL) and brine (30 mL). The organic phases were collected, dried over Na₂SO₄, filtered and concentrated. Flash chromatography (hexane/ethyl acetate, 1:1) then afforded the title compound (1.8 g, 67%): LCMS (ES) m/z385 (M+H)⁺; ¹H-NMR(CDCl₃) δ1.01 (m, 2H), 1.42 (m, 4H), 1.46 (s, 9H), 1.63 (m, 2H), 1.85 (m, 3H), 2.12 (m, 2H), 2.42 (m, 1H), 3.00 (m, 2H), 4.58 (s, br, 1H), 5.19 (s, br, 1H), 5.60 (s, br, 2H), 7.17 (m, 2H), 7.27 (m, 2H).

### 12b) Preparation of 1-(trans-4-aminomethyl-cyclohexyl)-1,2,3,4-tetrahydro-1,4-epiazano-naphthalen-9-yl-methanone

To a solution of [trans-4-(1,2,3,4-tetrahydro-1,4-epiazano-naphthalen-9-yl-methanoyl)-cyclohexylmethyl]-carbamic acid tert-butyl ester (0.95 g, 2.5 mmol) in CH₂Cl₂ (20 mL), trifluoroacetic acid (1.9 mL, 25 mmol) was added. The solution was stirred at room temperature for 4 hours before CH₂Cl₂ (30 mL) was added followed by Et₃N (5 mL). The resultant mixture was then washed with H₂O (30 mL), NaOH (1N, 30 mL) and brine (30 mL). The organic phases were collected, dried over K₂CO₃, filtered and concentrated to afford the title compound (0.57 g, 82%); LCMS (ES) m/z285 (M+H)⁺; ¹H-NMR(CDCl₃) δ 0.95 (m, 2H), 1.42 (m, 4H), 1.58 (m, 2H), 1.83 (m, 3H), 2.13 (m, 2H), 2.32 (m, 1H), 2.56 (m, 2H), 5.19 (s, 1H), 5.58 (s, 2H), 7.17 (m, 2H), 7.27 (m, 2H).

### 12c) Preparation of C-{trans-4-[1-(1,2,3,4-Tetrahydro-1,4-epiazano-naphthalen-9-yl)methyl]-cyclohexyl}-methylamine

To a solution of 1-(trans-4-aminomethyl-cyclohexyl)-1,2,3,4-tetrahydro-1,4-epiazano-naphthalen-9-yl-methanone (0.25 g, 0.88 mmol) in THF (2.0 mL), lithium aluminumhydride (1.0 N in THF, 2.6 mL, 2.6 mmol) was added. The solution was heated with a microwave reactor at 80° C. for 60 minutes before it was mixed with saturated aqueous Na₂SO₄ solution. The resultant mixture was filtered through celite. The organic phases were collected, dried over K₂CO₃, filtered and concentrated to afford the title compound (0.21 g, 88%): LCMS (ES) m/z271 (M+H)⁺; ¹H-NMR(CDCl₃) δ 0.88 (m, 4H), 1.30 (m, 6H), 1.82 (m, 4H), 1.97 (m, 2H), 2.12 (m, 2H), 2.52 (m, 2H), 4.15 (s, 2H), 7.13 (m, 2H), 7.22 (m, 2H).

### 12d) Preparation of quinoline-5-carboxylic acid {trans-4-[1-(1,2,3,4-tetrahydro-1,4-epiazano-naphthalen-9-yl)methyl]-cyclohexylmethyl}-amide

A solution of C{trans-4-[1-(1,2,3,4-tetrahydro-1,4-epiazano-naphthalen-9-yl)methyl]-cyclohexyl}-methylamine (30 mg, 0.111 mmol) in CH₂Cl₂ (2.0 mL) was mixed with 5-quinolinecarboxylic acid (21.1 mg, 0.122 mmol), EDC (23.4 mg, 0.122 mmol), HOBt (1.5 mg, 0.011 mmol) and Et₃N (0.109 mL, 0.777 mmol). The solution was stirred for 20 hours and concentrated. The resultant residue was dissolved in DMSO and purification via a reverse phase HPLC then afforded the title compound (67.5 mg, 93%): LCMS (ES) m/z426 (M+H)⁺; ¹H-NMR(CDCl₃) δ 1.05 (m, 4H), 1.61 (m, 4H), 1.84 (m, 4H), 2.63 (m, 4H), 3.40 (m, 2H), 5.07 (s, 2H), 7.44 (s, 4H), 8.01 (m, 3H), 8.54 (m, 1H), 9.17 (m, 1H), 9.52 (d, 1H).

### Example 13

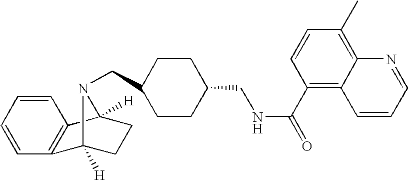

### 8-Methyl-quinoline-5-carboxylic acid {trans-4-[1-(1,2,3,4-tetrahydro-1,4-epiazano-naphthalen-9-yl)methyl]-cyclohexylmethyl}-amide

The title compound was prepared from C-{trans-4-[1-(1,2,3,4-tetrahydro-1,4-epiazano-naphthalen-9-yl)methyl]-cyclohexyl}-methylamine and 8-methyl-quinoline-5-carboxylic acid by following the experimental procedure in Example 12d (97% yield): LCMS (ES) m/z 440 (M+H)⁺; ¹H-NMR(CDCl₃) δ 1.04 (m, 4H), 1.61 (m, 4H), 1.85 (m, 4H), 2.63 (m, 2H), 2.72 (m, 2H), 2.95 (s, 3H), 3.40 (m, 2H), 5.05 (s, 2H), 7.44 (s, 4H), 7.87 (d, 1H), 7.99 (m, 2H), 9.33 (m, 1H), 9.60 (d, 1H).

### Example 14

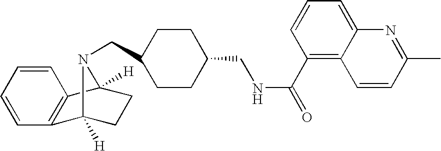

### 2-Methyl-quinoline-5-carboxylic acid {trans-4-[(1S,4S)-1-(1,2,3,4-tetrahydro-1,4-epiazano-naphthalen-9-yl)methyl]-cyclohexylmethyl}-amide

The title compound was prepared from C-{trans-4-[1-(1,2,3,4-tetrahydro-1,4-epiazano-naphthalen-9-yl)methyl]-cyclohexyl}-methylamine and 2-methyl-quinoline-5-carboxylic acid by following the experimental procedure in Example 12d (77% yield): LCMS (ES) m/z 440 (M+H)⁺; ¹H-NMR(CDCl₃) δ 1.01 (m, 4H), 1.61 (m, 4H), 1.83 (m, 4H), 2.65 (m, 4H), 3.07 (s, 3H), 3.38 (m, 2H), 5.07 (s, 2H), 7.45 (s, 4H), 7.73 (d, 1H), 7.96 (m, 2H), 8.45 (m, 1H), 9.35 (d, 1H).

### Example 15

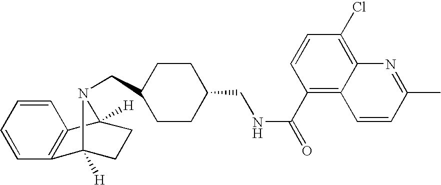

### 8-Chloro-2-methyl-quinoline-5-carboxylic acid {trans-4-[1-(1,2,3,4-tetrahydro-1,4-epiazano-naphthalen-9-yl)methyl]-cyclohexylmethyl}-amide

The title compound was prepared from C-{trans-4-[1-(1,2,3,4-tetrahydro-1,4-epiazano-naphthalen-9-yl)methyl]-cyclohexyl}-methylamine and 8-chloro-2-methyl-quinoline-5-carboxylic acid by following the experimental procedure in Example 12d (79% yield): LCMS (ES) m/z 474 (M+H)⁺; ¹H-NMR(CDCl₃) δ 1.00 (m, 4H), 1.59 (m, 4H), 1.82 (m, 4H), 2.60 (m, 2H), 2.68 (m, 2H), 3.02 (s, 3H), 3.35 (m, 2H), 5.05 (s, 2H), 7.43 (s, 4H), 7.66 (d, 1H), 7.76 (d, 1H), 7.88 (d, 1H), 9.07 (d, 1H).

### Example 16

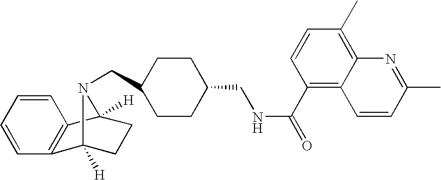

### 2,8-Dimethyl-quinoline-5-carboxylic acid {trans-4-[1-(1,2,3,4-tetrahydro-1,4-epiazano-naphthalen-9-yl)methyl]-cyclohexylmethyl}-amide

The title compound was prepared from G{trans-4-[1-(1,2,3,4-tetrahydro-1,4-epiazano-naphthalen-9-yl)methyl]-cyclohexyl}-methylamine and 2,8-dimethyl-quinoline-5-carboxylic acid by following the experimental procedure in Example 12d (83% yield): LCMS (ES) m/z454 (M+H)⁺; ¹H-NMR(CDCl₃) δ 1.05 (m, 4H), 1.62 (m, 4H), 1.84 (m, 4H), 2.76 (m, 4H), 2.91 (s, 3H), 3.13 (s, 3H), 3.38 (m, 2H), 5.06 (s, 2H), 7.43 (s, 4H), 7.81 (m, 2H), 9.43 (m, 1H), 10.44 (m, 1H).

### Example 17

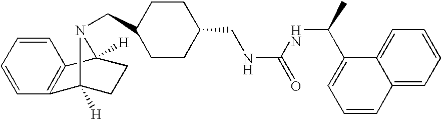

### 1-((S)-1-Naphthalen-1-yl-ethyl)-3-{trans-4-[1-(1,2,3,4-tetrahydro-1,4-epiazano-naphthalen-9-yl)methyl]-cyclohexylmethyl}-urea

A solution of C-{trans-4-[1-(1,2,3,4-tetrahydro-1,4-epiazano-naphthalen-9-yl)methyl]-cyclohexyl}-methylamine (30 mg, 0.111 mmol) in CH₂Cl₂ (2.0 mL) was mixed with 1-[(1S)-1-isocyanatoethyl]naphthalene (24 mg, 0.122 mmol) and stirred at room temperature for 3 hours. The solution was concentrated, redissolved in DMSO and filtered. Purification via a reverse phase HPLC then afforded the title compound (39.2 mg, 76%): LCMS (ES) m/z468 (M+H)⁺; ¹H-NMR(CDCl₃) δ 0.72 (m, 4H), 1.02 (m, 2H), 1.51 (m, 8H), 1.66 (d, 3H), 2.45 (m, 2H), 2.64 (m, 3H), 2.81 (m, 1H), 2.92 (m, 1H), 4.84 (s, 1H), 4.90 (s, 1H), 5.45 (s, br, 1H), 7.40 (s, 4H), 7.52 (m, 4H), 7.82 (d, 1H), 7.91 (d, 1H), 8.10 (d, 1H).

### Example 18

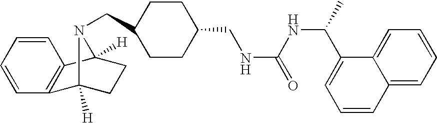

### 1-((R)-1-Naphthalen-1-yl-ethyl)-3-{trans-4-[(1-(1,2,3,4-tetrahydro-1,4-epiazano-naphthalen-9-yl)methyl]-cyclohexylmethyl}-urea

The title compound was prepared from C-{trans-4-[1-(1,2,3,4-tetrahydro-1,4-epiazano-naphthalen-9-yl)methyl]-cyclohexyl}-methylamine and 1-[(1S)-1-isocyanatoethyl]naphthalene by following the experimental procedure in Example 17 (80% yield): LCMS (ES) m/z468 (M+H)⁺; ¹H-NMR(CDCl₃) δ 0.71 (m, 4H), 1.00 (m, 2H), 1.55 (m, 8H), 1.68 (d, 3H), 2.49 (m, 2H), 2.67 (m, 3H), 2.81 (m, 1H), 2.93 (m, 1H), 4.92 (s, 1H), 4.94 (s, 1H), 5.41 (s, br, 1H), 7.41 (s, 4H), 7.53 (m, 4H), 7.83 (d, 1H), 7.92 (d, 1H), 8.08 (d, 1H).

### Example 19

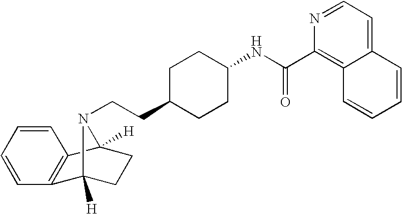

### Isoquinoline-1-carboxylic acid {trans-4-[(1S,4R)-2-(1,2,3,4-tetrahydro-1,4-epiazano-naphthalen-9-yl)-ethyl]-cyclohexyl}-amide

A solution o{trans-4-[2-(1,2,3,4-tetrahydro-1,4-epiazano-naphthalen-9-yl)-ethyl]-cyclohexylamine (40 mg, 0.15 mmol) in CHCl₃ (5 mL) was mixed with isoquinoline-1-carboxylic acid (28 mg, 0.16 mmol), EDC (28 mg, 0.15 mmol), HOBT (2 mg, 0.015 mmol) and DIEA (0.129 mL, 0.742 mmol). The resultant mixture was stirred at room temperature overnight, filtered and concentrated. Purification via a reverse phase HPLC then afforded the title compound (6 mg, 10%): LCMS (ES) m/z426 (M+H)⁺; ¹H-NMR(CDCl₃) δ 1.1 (m, 2H), 1.3 (m, 4H), 1.4 (m, 2H), 1.8 (m, 2H), 2.12 (m, 2H), 2.28 (m, 4H), 2.42 (m, 1H), 3.94 (m, 1H), 4.30 (s, 2H), 7.19 (m, 2H), 7.25 (m, 1H), 7.70 (m, 2H), 7.78 (d, 1H), 7.86 (d, 1H), 7.99 (d, 1H), 8.4(d, 1H), 9.6 (d, 1H).

### Example 20

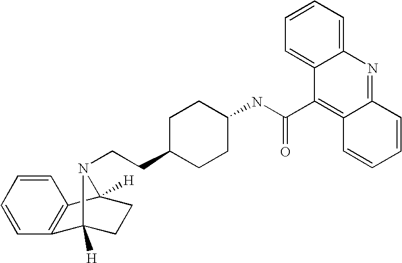

### Acridine-9-carboxylic acid {trans-4-[2-(1,2,3,4-tetrahydro-1,4-epiazano-naphthalen-9-yl)-ethyl]-cyclohexyl}-amide

The title compound was prepared from trans-4-[2-(1,2,3,4-tetrahydro-1,4-epiazano-naphthalen-9-yl)-ethyl]-cyclohexylamine and acridine-9-carboxylic acid by following the procedures in Example 19 (7% yield): LCMS (ES) m/z 476 (M+H)⁺; ¹H-NMR(CDCl₃) δ 0.9 (m, 2H), 1.31 (m, 7H), 1.47 (m, 2H), 1.81 (d, 2H), 2.20 (m, 2H), 2.28 (m, 2H), 4.26 (m, 3H), 6.10 (d, 1H), 7.22 (m, 2H), 7.31 (m, 2H), 7.55 (m, 2H), 7.77 (m, 2H), 8.02(d, 2H), 8.18 (d, 2H).

### Example 21

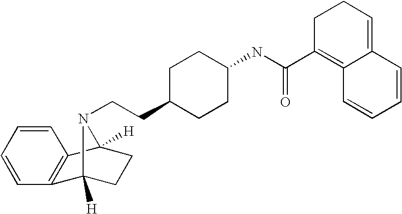

### 2,3-Dihydro-naphthalene-1-carboxylic acid {trans-4-[2-(1,2,3,4-tetrahydro-1,4-epiazano-naphthalen-9-yl)-ethyl]-cyclohexyl}-amide

A solution of {trans-4-[2-(1,2,3,4-tetrahydro-1,4-epiazano-naphthalen-9-yl)-ethyl]-cyclohexylamine (60 mg, 0.22 mmol) in CHCl₃ (3 mL) was mixed with naphthalene-1-carbonyl chloride (0.037 mL, 0.24 mmol) and Et₃N (0.1 mL, 0.72 mmol). The resultant mixture was stirred at room temperature overnight and concentrated. Purification via a reverse phase HPLC then afforded the title compound (34 mg, 36%): LCMS (ES) m/z 425 (M+H)⁺; ¹H-NMR(CDCl₃) δ 1.22 (m, 6H), 1.57 (m, 3H), 1.69 (m, 2H), 2.10 (d, 2H), 2.62 (m, 2H), 2.77 (m, 2H), 3.96 (m, 1H), 5.03 (s, 2H), 6.01 (d, 1H), 7.43 (m, 5H), 7.53 (m, 3H), 7.88 (m, 2H), 8.21 (d, 1H).

### Example 22

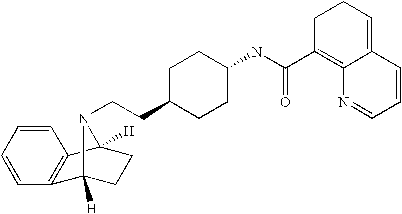

### 6,7-Dihydro-quinoline-8-carboxylic acid {trans-4-[2-(1,2,3,4-tetrahydro-1,4-epiazano-naphthalen-9-yl)-ethyl]-cyclohexyl}-amide

The title compound was prepared from trans-4-[2-(1,2,3,4-tetrahydro-1,4-epiazano-naphthalen-9-yl)-ethyl]-cyclohexylamine and quinoline-8-carboxylic acid by following the procedures in Example 19 (44%): LCMS (ES) m/z426 (M+H)⁺; ¹H-NMR(CDCl₃) δ 1.33 (m, 9H), 1.77 (d, 2H), 2.18 (m, 6H), 4.04 (m, 1H), 4.21 (s, 2H), 7.18 (m, 2H), 7.25 (m, 2H), 7.48 (t, 1H), 7.67 (t, 1H), 7.95 (d, 1H), 8.28 (d, 1H), 8.90 (m, 2H).

### Example 23

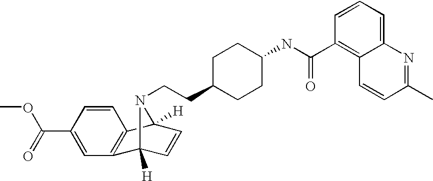

### 9-[2-(trans-4-{[1-(2-Methyl-quinolin-5-yl)-methanoyl]-amino}-cyclohexyl)-ethyl]-1,4-dihydro-1,4-epiazano-naphthalene-6-carboxylic acid methyl ester

### 23a) Preparation of 1,4-dihydro-1,4-epiazano-naphthalene-6,9-dicarboxylic acid 9-tert-butyl ester 6-methyl ester

A solution of [5-(methoxycarbonyl)-2-(trimethylsilyl)phenyl]-(phenyl)iodonium triflate (7.1 g, 12.7 mmol) and pyrrole-1-carboxylic acid tert-butyl ester (10.6 mL, 63.4 mmol) in CH₂Cl₂ (100 mL) was cooled to 0° C. and added by a THF solution of Bu₄NF (16.5 mL, 1 M in THF, 16.5 mmol). The mixture was stirred for 30 min. Water was added to the mixture, and the product was extracted with CH₂Cl₂. The combined organic phases were collected, dried over MgSO₄ and concentrated. Flash chromatography then afforded the title compound (3.0 g, 78.7%): LCMS (ES) m/z302 (M+H)⁺; ¹H-NMR(CDCl₃) δ 1.37 (s, 9H), 3.89 (s, 3H), 5.52 (s, br, 2H), 6.89 (s, br, 2H), 7.34 (m, 1H), 7.71 (m, 1H), 7.89 (s, 1H).

### 23b) Preparation of 1,4-ddihydro-1,4-epiazano-naphthalene-6-carboxylic acid methyl ester

A solution of 1,4-dihydro-1,4-epiazano-naphthalene-6,9-dicarboxylic acid 9-tert-butyl ester 6-methyl ester (1 g, 3.31 mmol) in CH₂Cl₂ (40 mL) was mixed with TFA (2.25 mL, 29.2 mmol) at 0° C. The mixture was stirred at room temperature overnight, diluted with EtOAc (15 mL) and concentrated. The resultant residue was extracted with aqueous NaOH solution (2N, 20 mL), H₂O (50 mL) and brine (50 mL). The organic phases were collected, dried over Na₂SO₄ and concentrated to afford the title compound (0.5 g, 75%): LCMS (ES) m/z202 (M+H)⁺.

### 23c) Preparation of 9-[2-(trans-4-tert-Butoxycarbonylamino-cyclohexyl)-ethyl]-1,4-dihydro-1,4-epiazano-naphthalene-6-carboxylic acid methyl ester

A solution of 1,4-dihydro-1,4-epiazano-naphthalene-6-carboxylic acid methyl ester (0.5 g, 2.48 mmol) in 1,2-dichloroethane (25 mL) was mixed with 3,3-dimethyl-N-[4-(2-oxo-ethyl)-cyclohexyl]-butyramide (0.6 g, 2.49 mmol) and NaB(OAc)₃H (0.84 g, 3.96 mmol). The mixture was stirred at room temperature overnight and diluted with CH₂Cl₂ (30 mL). The solution was extracted with saturated aqueous NaHCO₃ solution (50 mL) and brine (50 mL). The organic phases were collected, dried over MgSO₄, and concentrated to afford the title compound (0.9 g, 85%); MS (ES) m/z427 (M+H)⁺.

### 23d) Preparation of 9-[2-(trans-4-amino-cyclohexyl)-ethyl]-1,4-dihydro-1,4-epiazano-naphthalene-6-carboxylic acid methyl ester

The title compound was prepared from 9-[2-(trans-4-tert-butoxycarbonylamino-cyclohexyl)-ethyl]-1,4-dihydro-1,4-epiazano-naphthalene-6-carboxylic acid methyl ester by following the procedure in Example 23b (36%): LCMS (ES) m/z327 (M+H)⁺.

### 23e) Preparation of 9-[2-(trans-4-{[1-(2-methyl-quinolin-5-yl)-methanoyl]-amino}-cyclohexyl)-ethyl]-1,4-dihydro-1,4-epiazano-naphthalene-6-carboxylic acid methyl ester

The title compound was prepared from 9-[2-(trans-4-amino-cyclohexyl)-ethyl]-1,4-dihydro-1,4-epiazano-naphthalene-6-carboxylic acid methyl ester and quinoline-5-carboxylic acid by following the procedures in Example 19 (73% yield): LCMS (ES) m/z496 (M+H)⁺; ¹H-NMR(CDCl₃) δ1.19 (m, 5H), 1.58 (m, 2H), 1.71 (m, 2H), 2.14 (m, 2H), 2.87 (m, 2H), 3.07 (s, 3H), 3.97 (m, 4H), 5.52 (m, 2H), 6.35 (d, 1H), 7.15 (m, 2H), 7.61 (d, 1H), 7.69 (d, 1H), 7.95 (m, 2H), 8.06 (d, 2H), 8.15 (s, 1H), 8.59 (d, 1H), 9.35 (d, 1H).

### Example 24

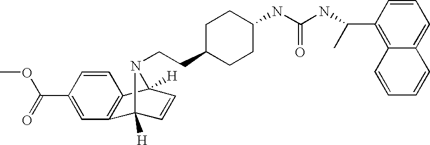

### 9-(2-{trans-4-[3-((S)-1-Naphthalen-1-yl-ethyl)-ureido]-cyclohexyl}-ethyl)-1,4-dihydro-1,4-epiazano-naphthalene-6-carboxylic acid methyl ester

The title compound was prepared from 9-[2-(trans-4-amino-cyclohexyl)-ethyl]-1,4-dihydro-1,4-epiazano-naphthalene-6-carboxylic acid methyl ester and 1-((S)-1-isocyanato-ethyl)-naphthalene by following the procedure in Example 17 (50%): LCMS (ES) m/z524 (M+H)⁺; ¹H-NMR(CDCl₃) δ 0.90 (m, 3H), 1.27 (m, 2H), 1.50 (m, 4H), 1.65 (d, 3H), 1.75 (m, 1H), 1.88 (m, 1H), 2.71 (m, 2H), 3.35 (m, 1H), 3.91 (m, 1H), 3.96 (s, 3H), 4.40 (s, br, 1H), 5.35 (m, 2H), 5.55 (m, 1H), 7.03 (m, 2H), 7.55 (m, 5H), 7.78 (m, 1H), 7.88 (m, 1H), 8.00 (m, 1H), 8.07 (d, 1H), 8.13 (d, 1H).

### Example 25

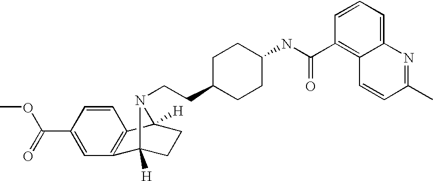

### 9-[2-(trans-4-{[1-(2-Methyl-quinolin-5-yl)-methanoyl]-amino}-cyclohexyl)-ethyl]-1,2,3,4-tetrahydro-1,4-epiazano-naphthalene-6-carboxylic acid methyl ester

### 25a) Preparation of 9-[2-(trans-4-tert-butoxycarbonylamino-cyclohexyl)-ethyl]-1,2,3,4-tetrahydro-1,4-epiazano-naphthalene-6-carboxylic acid methyl ester

A solution of 9-[2-(trans-4-amino-cyclohexyl)-ethyl]-1,4-dihydro-1,4-epiazano-naphthalene-6-carboxylic acid methyl ester (0.4 g, 0.94 mmol) in ethanol (5 mL) was mixed with 10% Pd/C (45 mg). The mixture was stirred in the presence of H₂ (55 psi) at room temperature for 3 hours, filtered through celite and concentrated to afford the title compound (0.24 g, 60%): LCMS (ES) m/z 429 (M+H)⁺.

### 25b) Preparation of 9-[2-(trans-4-amino-cyclohexyl)-ethyl]-1,2,3,4-tetrahydro-1,4-epiazano-naphthalene-6-carboxylic acid methyl ester

The title compound was prepared from 9-[2-(trans-4-tert-butoxycarbonylamino-cyclohexyl)-ethyl]-1,2,3,4-tetrahydro-1,4-epiazano-naphthalene-6-carboxylic acid methyl ester by following the procedures in Example 23b (60%): LCMS (ES) m/z329 (M+H)⁺.

### 25c) Preparation of 9-[2-(trans-4-{[1-(2-methyl-quinolin-5-yl)-methanoyl]-amino)-cyclohexyl)-ethyl]-1,2,3,4-tetrahydro-1,4-epiazano-naphthalene-6-carboxylic acid methyl ester

The title compound was prepared from 9-[2-(trans-4-amino-cyclohexyl)-ethyl]-1,2,3,4-tetrahydro-1,4-epiazano-naphthalene-6-carboxylic acid methyl ester and 2-methyl-quinoline-5-carboxylic acid by following the procedures in Example 19 (26% yield): LCMS (ES) m/z498 (M+H)⁺; ¹H-NMR(CDCl₃) δ1.08 (m, 2H), 1.27 (m, 3H), 1.60 (m, 4H), 1.73 (m, 2H), 2.11 (m, 2H), 2.71 (m, 2H), 2.79 (m, 2H), 3.05 (s, 3H), 3.94 (m, 1H), 3.98 (s, 3H), 5.12 (s, 2H), 6.6 (m, 1H), 7.56 (d, 1H), 7.69 (d, 1H), 7.93 (m, 2H), 8.12 (s, 1H), 8.18 (d, 1H), 8.50 (s, 1H), 9.35 (d, 1H).

### Example 26

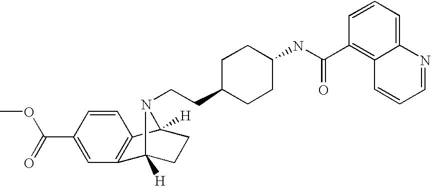

### (1S,4R)-9-(2-{4-[(1-Quinolin-5-yl-methanoyl)-amino]-cyclohexyl}-ethyl)-1,2,3,4-tetrahydro-1,4-epiazano-naphthalene-6-carboxylic acid methyl ester

The title compound was prepared from 9-[2-(trans-4-amino-cyclohexyl)-ethyl]-1,2,3,4-tetrahydro-1,4-epiazano-naphthalene-6-carboxylic acid methyl ester and quinoline-5-carboxylic acid by following the procedures in Example 19 (92% yield): LCMS (ES) m/z484 (M+H)⁺; ¹H-NMR(CDCl₃) δ1.09 (m, 2H), 1.27 (m, 3H), 1.58 (m, 4H), 1.72 (m, 2H), 2.14 (m, 2H), 2.72 (m, 2H), 2.78 (m, 2H), 3.94 (m, 1H), 3.97 (s, 3H), 5.12 (s, 2H), 6.37 (d, 1H), 7.56 (d, 1H), 7.95 (m, 3H), 8.12 (m, 1H), 8.18 (d, 1H), 8.56 (d, 1H), 9.23 (d, 1H), 9.43 (d, 1H).

### Example 27

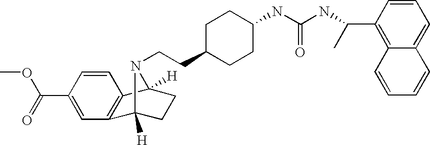

### 9-(2-{trans-4-[3-((S)-1-Naphthalen-1-yl-ethyl)-ureido]-cyclohexyl}-ethyl)-1,2,3,4-tetrahydro-1,4-epiazano-naphthalene-6-carboxylic acid methyl ester

The title compound was prepared from 9-[2-(trans-4-amino-cyclohexyl)-ethyl]-1,2,3,4-tetrahydro-1,4-epiazano-naphthalene-6-carboxylic acid methyl ester and 1-((S)-1-isocyanato-ethyl)-naphthalene by following the procedure in Example 17 (41% yield): LCMS (ES) m/z526 (M+H)⁺; ¹H-NMR(CDCl₃) δ 0.89 (m, 5H), 1.07 (m, 1H), 1.27 (m, 2H), 1.49 (m, 5H), 1.64 (m, 3H), 1.79 (m, 1H), 1.88 (m, 1H), 2.66 (m, 4H), 3.37 (m, 1H), 3.97 (s, 3H), 4.92 (m, 2H), 5.59 (m, 1H), 7.55 (m, 4H), 7.77 (d, 1H), 7.89 (d, 1H), 8.06 (d, 1H), 8.15 (m, 2H).

### Example 28

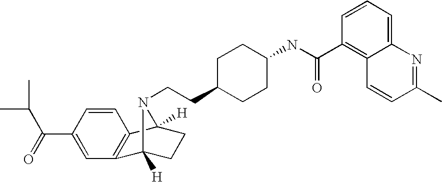

### 2-Methyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide

### 28a) Preparation of 2-ethyl-1-1,2,3,4-tetrahydro-1,4-epiazano-naphthalen-6-yl-propan-1-one

The title compound was prepared from 6-(2-methyl-propanoyl)-1,2,3,4-tetrahydro-1,4-epiazano-naphthalene-9-carboxylic acid tert-butyl ester by following the procedure in Example 23b (77% yield): LCMS (ES) m/z431 (2M+H)⁺.

### 28b) Preparation of (trans-4-{2-[6-(2-methyl-propanoyl)-1,2,3,4-tetrahydro-1,4-epiazano-naphthalen-9-yl]-ethyl}-cyclohexyl)-carbamic acid tert-butyl ester

The title compound was prepared from 2-ethyl-1-1,2,3,4-tetrahydro-1,4-epiazano-naphthalen-6-yl-propan-1-one by following the procedures in Example 23c (39% yield): LCMS (ES) m/z 441 (M+H)⁺.

### 28c) Preparation of 1-{9-[2-(trans-4-amino-cyclohexyl)-ethyl]-1,2,3,4-tetrahydro-1,4-epiazano-naphthalen-6-yl}-2-methyl-propan-1-one

The title compound was prepared from (trans-4-{2-[6-(2-methyl-propanoyl)-1,2,3,4-tetrahydro-1,4-epiazano-naphthalen-9-yl]-ethyl}-cyclohexyl)-carbamic acid tert-butyl ester by following the procedure in Example 23b (97% yield): LCMS (ES) m/z 341 (M+H)⁺.

### 28d) Preparation of 2-methyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-methyl-propan-1-one and 2-methyl-quinoline-5-carboxylic acid by following the procedures in Example 19 (69% yield): LCMS (ES) m/z510 (M+H)⁺; ¹H-NMR(CDCl₃) δ1.09 (m, 2H), 1.27 (m, 8H), 1.61 (m, 4H), 1.74 (m, 2H), 2.13 (m, 2H), 2.38 (m, 1H), 2.71 (m, 2H), 2.81 (m, 2H), 3.06 (s, 3H), 3.56 (m, 1H), 4.0 (m, 1H), 5.14 (s, 2H), 6.48 (d, 1H), 7.20 (m, 1H), 7.58 (d, 1H), 7.70 (d, 1H), 7.95 (m, 2H), 8.06 (d, 1H), 8.53 (d, 1H), 9.37 (d, 1H).

### Example 29

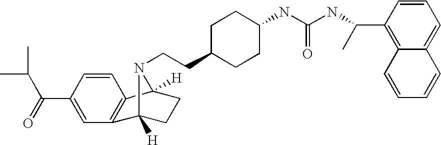

### 1-(trans-4-[2-[6-(2-Methyl-propanoyl)-1,2,3,4-tetrahydro-1,4-epiazano-naphthalen-9-yl]-ethyl}-cyclohexyl)-3-((S)-1-naphthalen-1-yl-ethyl)-urea

The title compound was prepared from 1-{9-[2-(trans-4-amino-cyclohexyl)-ethyl]-1,2,3,4-tetrahydro-1,4-epiazano-naphthalen-6-yl}-2-methyl-propan-1-one and 1-((S)-1-isocyanato-ethyl)-naphthalene by following the procedure in Example 17 (51% yield): LCMS (ES) m/z538 (M+H)⁺; ¹H-NMR(CDCl₃) δ0.7 (m, 1H), 0.9 (m, 3H), 1.0 (m, 1H), 1.20 (m, 1H), 1.30 (m, 6H), 1.47 (m, 3H), 1.57 (m, 2H), 1.75 (d, 4H), 1.86 (m, 1H), 2.06 (m, 1H), 2.66 (d, 3H), 3.29 (m, 1H), 3.54 (m, 2H), 4.96 (s, 2H), 5.47 (d, 1H), 7.57 (m, 5H), 7.81 (m, 1H), 7.89 (d, 1H), 8.00 (m, 2H), 8.10 (d, 1H).

### Example 30

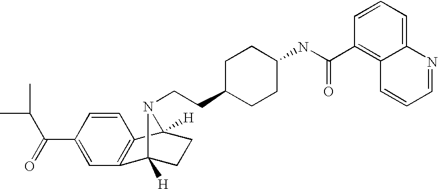

### Quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide

The title compound was prepared from 1-(9-[2-(trans-4-amino-cyclohexyl)-ethyl]-1,2,3,4-tetrahydro-1,4-epiazano-naphthalen-6-yl}-2-methyl-propan-1-one and quinoline-5-carboxylic acid by following the procedures in Example 19 (60% yield): LCMS (ES) m/z496 (M+H)⁺; ¹H-NMR(CDCl₃) δ1.10 (m, 2H), 1.20 (m, 1H), 1.31 (m, 8H), 1.61 (m, 4H), 1.73 (m, 2H), 2.12 (m, 2H), 2.72 (m, 2H), 2.80 (m, 2H), 3.56 (m, 1H), 3.97 (m, 1H), 5.13 (s, 2H), 6.43 (d, 1H), 7.58 (d, 1H), 7.95 (m, 3H), 8.07 (m, 2H), 8.56 (d, 1H), 9.23 (m, 1H), 9.46 (d, 1H).

### Example 31

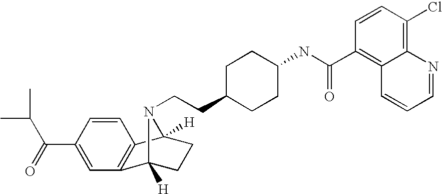

### 8-Chloro-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-methyl-propan-1-one and 8-chloro-quinoline-5-carboxylic acid by following the procedures in Example 19 (79% yield): LCMS (ES) m/z530 (M+H)⁺; ¹H-NMR(CDCl₃) δ 1.10 (m, 2H), 1.20 (m, 3H), 1.28 (d, 6H), 1.58 (m, 2H), 1.65 (m, 2H), 1.70 (d, 2H), 2.12 (m, 2H), 2.68 (m, 2H), 2.81 (d, 2H), 3.56 (m, 1H), 3.9 (m, 1H), 5.17 (s, 2H), 6.30 (d, 1H), 7.56 (m, 1H), 7.66 (m, 1H), 7.78 (m, 1H), 7.87 (m, 1H), 7.97 (m, 1H), 8.07 (m, 1H), 9.00 (m, 1H), 9.22 (m, 1H).

### Example 32

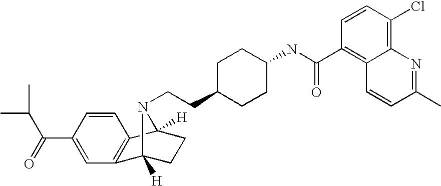

### 8-Chloro-2-methyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-methyl-propan-1-one and 8-chloro-2-methyl-quinoline-5-carboxylic acid by following the procedures in Example 19 (63% yield): LCMS (ES) m/z544 (M+H)⁺; ¹H-NMR(CDCl₃) δ 1.10 (m, 2H), 1.21 (m, 3H), 1.28 (d, 6H), 1.61 (m, 4H), 1.70 (m, 2H), 2.10 (m, 2H), 2.70 (m, 2H), 2.77 (m, 2H), 2.93 (s, 3H), 3.58 (m, 1H), 3.95 (m, 1H), 5.15 (s, 2H), 6.34 (d, 1H), 7.58 (m, 3H), 7.80 (d, 1H), 8.05 (m, 2H), 8.85 (d, 1H).

### Example 33

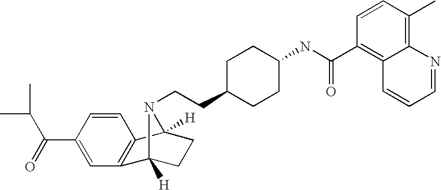

### 8-Methyl-quinoline-5-carboxylic acid (4-{2-[(1S,4R)-6-(2-methyl-propanoyl)-1,2,3,4-tetrahydro-1,4-epiazano-naphthalen-9-yl]-ethyl}-cyclohexyl]-amide

The title compound was prepared from 1-{9-[2-(trans-4-amino-cyclohexyl)-ethyl]-1,2,3,4-tetrahydro-1,4-epiazano-naphthalen-6-yl}-2-methyl-propan-1-one and 8-methyl-quinoline-5-carboxylic acid by following the procedures in Example 19 (74% yield): LCMS (ES) m/z510 (M+H)⁺; ¹H-NMR(CDCl₃) δ1.10 (m, 2H), 1.21 (m, 3H), 1.27 (d, 6H), 1.58 (m, 4H), 1.71 (m, 2H), 2.10 (m, 2H), 2.71 (m, 2H), 2.79 (m, 2H), 2.92 (s, 3H), 3.58 (m, 1H), 3.95 (m, 1H), 5.13 (s, 2H), 6.47 (d, 1H), 7.57 (d, 1H), 7.80 (m, 2H), 7.88 (m, 1H), 8.05 (d, 2H), 9.33 (d, 1H), 9.44 (d, 1H).

### Example 34

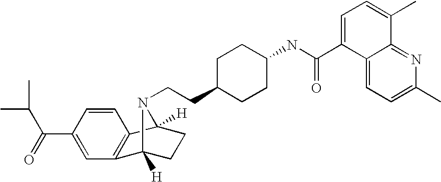

### 2,8-Dimethyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-methyl-propan-1-one and 2,8-dimethyl-quinoline-5-carboxylic acid by following the procedures in Example 19 (91% yield): LCMS (ES) m/z524 (M+H)⁺; ¹H-NMR(CDCl₃) δ 1.10 (m, 2H), 1.22 (m, 3H), 1.27 (d, 6H), 1.60 (m, 4H), 1.73 (m, 2H), 2.10 (m, 2H), 2.71 (m, 2H), 2.79 (m, 2H), 2.90 (s, 3H), 3.08 (s, 3H), 3.57 (m, 1H), 3.92 (m, 1H), 5.12 (s, 2H), 6.51 (d, 1H), 7.57 (d, 1H), 7.69 (m, 1H), 7.77 (m, 2H), 8.05 (d, 2H), 9.37 (d, 1H).

### Example 35

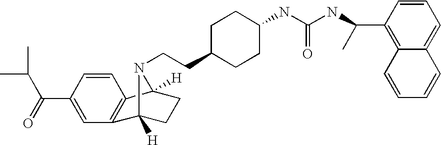

### 1-(trans-4-{2-[6-(2-Methyl-propanoyl)-1,2,3,4-tetrahydro-1,4-epiazano-naphthalen-9-yl]-ethyl}-cyclohexyl)-3-((R)-1-naphthalen-1-yl-ethyl)-urea

The title compound was prepared from 1-{9-[2-(trans-4-amino-cyclohexyl)-ethyl]-1,2,3,4-tetrahydro-1,4-epiazano-naphthalen-6-yl)-2-methyl-propan-1-one and 1-((R)-1-isocyanato-ethyl)-naphthalene by following the procedure in Example 17 (48% yield): LCMS (ES) m/z538 (M+H)⁺; ¹H-NMR(CDCl₃) δ 0.80 (m, 5H), 1.10 (m, 2H), 1.22 (m, 1H), 1.27 (d, 6H), 1.50 (m, 6H), 1.64 (m, 2H), 1.76 (m, 1H), 1.88 (m, 1H), 2.66 (s, 3H), 3.36 (m, 1H), 3.55 (m, 1H), 4.97 (m, 2H), 5.56 (d, 1H), 7.55 (m, 5H), 7.78 (m, 1H), 7.88 (d, 1H), 8.00 (m, 2H), 8.14 (d, 1H).

### Example 36

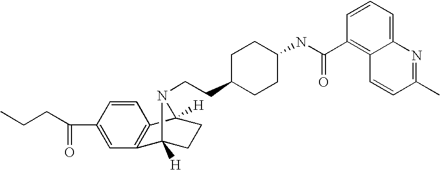

### 2-Methyl-quinoline-5-carboxylic acid (trans-4-{2-[6-butyryl-1,2,3,4-tetrahydro-1,4-epiazano-naphthalen-9-yl]-ethyl}-cyclohexyl)-amide

### 36a) Preparation of 1,2,3,4-tetrahydro-1,4-epiazano-naphthalene-6,9-dicarboxylic acid 9-tert-butyl ester 6-methyl ester

A solution of 1,4-dihydro-1,4-epiazano-naphthalene-6,9-dicarboxylic acid tert-butyl ester 6-methyl ester (0.47 g, 1.56 mmol) in ethanol (8 mL) was mixed with Pd/C (10%, 74 mg). The mixture was stirred in the presence of H₂ (55 psi) at room temperature overnight, filtered through celite and concentrated to afford the title compound (0.46 g, 98%): LCMS (ES) m/z 607 (2M+H)⁺.

### 36b) Preparation of 6-hydroxymethyl-1,2,3,4-tetrahydro-1,4-epiazano-naphthalene-9-carboxylic acid 9-tert-butyl ester

A solution of 1,2,3,4-tetrahydro-1,4-epiazano-naphthalene-6,9-dicarboxylic acid 9-tert-butyl ester 6-methyl ester (0.52 g, 1.71 mmol) in THF (20 mL) was mixed with super hydride (6.8 mL, 1.0M solution in THF, 6.8 mmol) at 0° C. The mixture was stirred at ambient temperature for 2 hours, mixed with HCl (10 ml, 1 N), diluted with brine (50 mL) and extracted with EtOAc (50 mL). The organic phases were collected, dried over Na₂SO₄ and concentrated to afford the title compound (0.47 g, 100%): LCMS (ES) m/z276 (M+H)⁺.

### 36c) Preparation of 6-formyl-1,2,3,4-tetrahydro-1,4-epiazano-naphthalene-9-carboxylic acid tert-butyl ester

A solution of 6-hydroxymethyl-1,2,3,4-tetrahydro-1,4-epiazano-naphthalene-9-carboxylic acid 9-tert-butyl ester (0.47 g, 1.70 mmol) in CH₂Cl₂ (20 mL) was mixed with Dess-Martin periodinane (0.75 g, 1.77 mmol). The mixture was stirred at r.t. for 2 hours and concentrated. Flash chromatography (Hexane:EtOAc, 4:1) then afforded the title compound (0.40 g, 85%): MS (ES) m/z274 (M+H)⁺.

### 36d) Preparation of 6-(1-hydroxy-butyl)-1,2,3,4-tetrahydro-1,4-epiazano-naphthalene-9-carboxylic acid tert-butyl ester

A solution of 6-formyl-1,2,3,4-tetrahydro-1,4-epiazano-naphthalene-9-carboxylic acid 9-tert-butyl ester (0.40 g, 1.46 mmol) in THF (20 mL) was mixed with propyl magnesium chloride (2.2 mL, 2.0M solution in THF, 4.4 mmol) at 0° C. The mixture was stirred at r.t. overnight, diluted with brine (50 mL) and extracted with EtOAc (50 mL). The combined organic phases were dried over Na₂SO₄ and concentrated. Purification via a combiflash system then afforded the title compound (0.33 g, 71%): LCMS (ES) m/z318 (M+H)⁺.

### 36e) Preparation of 6-butyryl-1,2,3,4-tetrahydro-1,4-epiazano-naphthalene-9-carboxylic acid tert-butyl ester

The title compound was prepared from 6-formyl-1,2,3,4-tetrahydro-1,4-epiazano-naphthalene-9-carboxylic acid 9-tert-butyl ester by following the procedures in Example 36c (91%): LCMS (ES) m/z316 (M+H)⁺.

### 36f) Preparation of 1-(1,2,3,4-tetrahydro-1,4-epiazano-naphthalen-6-yl)-butan-1-one

The title compound was prepared from 6-butyryl-1,2,3,4-tetrahydro-1,4-epiazano-naphthalene-9-carboxylic acid tert-butyl ester by following the procedure in Example 23b (63% yield): LCMS (ES) m/z431 (2M+H)⁺.

### 36g) Preparation of {trans-4-[2-(6-butyryl-1,2,3,4-tetrahydro-1,4-epiazano-naphthalen-9-yl)-ethyl]-cyclohexyl}-carbamic acid tert-butyl ester

The title compound was prepared from 1-(1,2,3,4-tetrahydro-1,4-epiazano-naphthalen-6-yl)-butan-1-one by following the procedures in Example 23c (57% yield): LCMS (ES) m/z441 (M+H)⁺.

### 36h) Preparation of 1-{9-[2-(trans-4-amino-cyclohexyl)-ethyl]-1,2,3,4-tetrahydro-1,4-epiazano-naphthalen-6-yl}-butan-1-one

The title compound was prepared from {trans-4-[2-(6-butyryl-1,2,3,4-tetrahydro-1,4-epiazano-naphthalen-9-yl)-ethyl]-cyclohexyl}-carbamic acid tert-butyl ester by following the procedures in Example 23b (98% yield): LCMS (ES) m/z341 (M+H)⁺.

### 36l) Preparation of methyl-quinoline-5-carboxylic acid (trans-4-{2-[6-butyryl-1,2,3,4-tetrahydro-1,4-epiazano-naphthalen-9-yl]-ethyl}-cyclohexyl)-amide

The title compound was prepared from 1-(9-[2-(trans-4-amino-cyclohexyl)-ethyl]-1,2,3,4-tetrahydro-1,4-epiazano-naphthalen-6-yl}-butan-1-one and 2-methyl-quinoline-5-carboxylic acid by following the procedures in Example 19 (53% yield): LCMS (ES) m/z510 (M+H)⁺; ¹H-NMR(CDCl₃) δ1.09 (m, 4H), 1.27 (m, 4H), 1.60 (m, 4H), 1.76 (m, 2H), 1.83 (m, 2H), 2.10 (m, 2H), 2.70 (m, 2H), 2.78 (m, 2H), 2.99 (m, 2H), 3.06 (s, 3H), 4.0 (m, 1H), 5.12 (s, 2H), 6.49 (d, 1H), 7.57 (d, 1H), 7.69 (d, 1H), 7.94 (m, 2H), 8.07 (m, 2H), 8.53 (d, 1H), 9.36 (d, 1H).

### Example 37

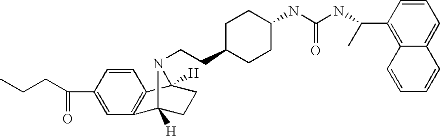

### 1-(trans-4-{2-[6-Butyryl-1,2,3,4-tetrahydro-1,4-epiazano-naphthalen-9-yl]-ethyl}-cyclohexyl)-3-((S)-1-naphthalen-1-yl-ethyl)-urea

The title compound was prepared from 1-{9-[2-(trans-4-amino-cyclohexyl)-ethyl]-1,2,3,4-tetrahydro-1,4-epiazano-naphthalen-6-yl}-butan-1-one and 1-((S)-1-isocyanato-ethyl)-naphthalene by following the procedure in Example 17 (63% yield): LCMS (ES) m/z538 (M+H)⁺; ¹H-NMR(CDCl₃) δ 0.65 (m, 1H), 0.85 (m, 3H), 1.06 (m, 4H), 1.28 (m, 2H), 1.48 (m, 2H), 1.59 (m, 4H), 1.71 (m, 3H), 1.81 (m, 3H), 2.03 (m, 1H), 2.65 (d, 3H), 2.97 (m, 2H), 3.25 (m, 1H), 5.03 (s, 2H), 5.40 (m, 1H), 7.56 (m, 5H), 7.84 (m, 1H), 7.91 (m, 1H), 8.06 (m, 3H).

### Example 38

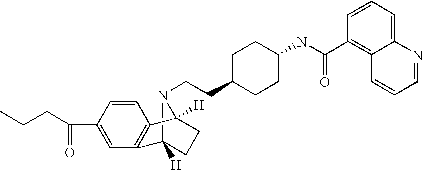

### Quinoline-5-carboxylic acid (trans-4-{2-[6-butyryl-propanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-butan-1-one and quinoline-5-carboxylic acid by following the procedures in Example 19 (34% yield): LCMS (ES) m/z496 (M+H)⁺; ¹H-NMR(CDCl₃) δ1.11 (m, 2H), 1.31 (m, 6H), 1.60 (m, 4H), 1.78 (m, 4H), 2.16 (m, 2H), 2.81 (m, 4H), 2.99 (t, 2H), 3.99 (m, 1H), 5.12 (s, 2H), 6.18 (d, 1H), 7.57 (m, 1H), 7.94 (m, 2H), 8.00 (m, 1H), 8.07 (m, 2H), 8.61 (d, 1H), 9.25 (d, 1H), 9.44 (d, 1H).

### Example 39

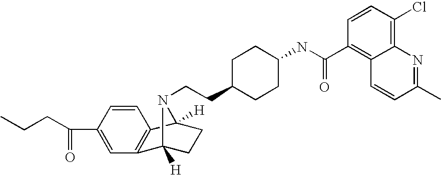

### 8-Chloro-2-methyl-quinoline-5-carboxylic acid (trans-4-{2-[6-butyryl-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-butan-1-one and quinoline-8-chloro-2-methyl-5-carboxylic acid by following the procedures in Example 19 (34% yield): LCMS (ES) m/z544 (M+H)⁺; ¹H-NMR(CDCl₃) δ1.10 (m, 4H), 1.22 (m, 4H), 1.61 (m, 4H), 1.73 (m, 2H), 1.85 (m, 2H), 2.14 (m, 2H), 2.76 (m, 4H), 2.85 (s, 3H), 2.99 (t, 2H), 3.95 (m, 1H), 5.09 (s, 2H), 5.90 (d, 1H), 7.44 (d, 1H), 7.50 (d, 1H), 7.56 (d, 1H), 7.78 (d, 1H), 8.07 (m, 2H), 8.66 (d, 1H).

### Example 40

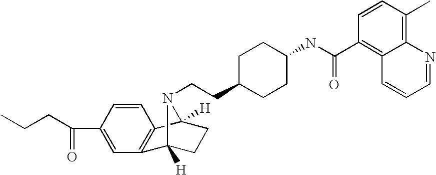

### 8-Methyl-quinoline-5-carboxylic acid (4-{2-[6-butyryl-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-butan-1-one and quinoline-8-methyl-5-carboxylic acid by following the procedures in Example 19 (87% yield): LCMS (ES) m/z510 (M+H)⁺; ¹H-NMR(CDCl₃) δ1.12 (m, 4H), 1.27 (m, 4H), 1.59 (m, 4H), 1.75 (m, 2H), 1.83 (m, 2H), 2.12 (m, 2H), 2.70 (m, 2H), 2.79 (m, 2H), 2.94 (s, 3H), 3.01 (t, 2H), 3.97 (m, 1H), 5.12 (s, 2H), 6.22 (d, 1H), 7.55 (d, 1H), 7.77 (m, 2H), 7.87 (m, 1H), 8.07 (m, 2H), 9.39 (t, 2H).

### Example 41

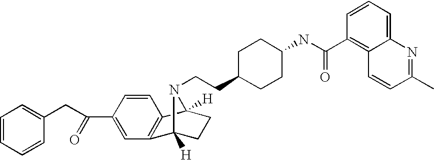

### 2-Methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide

### 41a) Preparation of 1-{9-[2-(trans-4-amino-cyclohexyl)-ethyl]-1,2,3,4-tetrahydro-1,4-epiazano-naphthalen-6-yl}-2-phenyl-ethanone

The title compound was prepared from 6-formyl-1,2,3,4-tetrahydro-1,4-epiazano-naphthalene-9-carboxylic acid tert-butyl ester and PhCH₂MgBr by following the procedures in 36d, 36e, 36f, 36g and 36h: LCMS (ES) m/z 389 (M+H)⁺.

### 41b) Preparation of 2-methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and quinoline-2-methyl-5-carboxylic acid by following the procedures in Example 19 (75% yield): LCMS (ES) m/z558 (M+H)⁺, ¹H-NMR(CDCl₃) δ 1.07 (m, 1H), 1.27 (m, 4H), 1.57 (m, 4H), 1.69 (m, 2H), 2.08 (m, 2H), 2.70 (m, 2H), 2.77 (m, 2H), 3.04 (s, 3H), 3.95 (m, 1H), 4.32 (s, 2H), 5.11 (s, 2H), 6.70 (d, 1H), 7.28 (m, 3H), 7.39 (m, 2H), 7.54 (d, 1H), 7.67 (d, 1H), 7.89 (m, 2H), 8.12 (m, 2H), 8.48 (m, 1H), 9.32 (d, 1H).

### Example 42

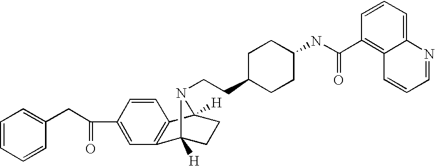

### Quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and quinoline-5-carboxylic acid by following the procedures in Example 19 (83% yield): LCMS (ES) m/z544 (M+H)⁺, ¹H-NMR(CDCl₃) δ1.07 (m, 1H), 1.27 (m, 4H), 1.57 (m, 4H), 1.73 (m, 2H), 2.11 (m, 2H), 2.70 (m, 2H), 2.77 (m, 2H), 3.96 (m, 1H), 4.32 (s, 2H), 5.12 (s, 2H), 6.48 (d, 1H), 7.30 (m, 3H), 7.39 (m, 2H), 7.55 (d, 1H), 7.93 (m, 3H), 8.13 (m, 2H), 8.54 (d, 1H), 9.21 (d, 1H), 9.42 (d, 1H).

### Example 43

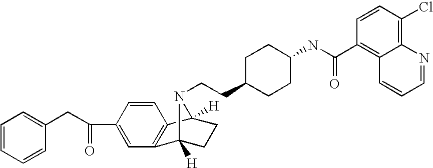

### 8-Chloro-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and quinoline-8-chloro-carboxylic acid by following the procedures in Example 19 (81% yield): LCMS (ES) m/z578 (M+H)⁺; ¹H-NMR(CDCl₃) δ1.07 (m, 1H), 1.24 (m, 4H), 1.57 (m, 4H), 1.71 (m, 2H), 2.13 (m, 2H), 2.67 (m, 2H), 2.77 (m, 2H), 3.96 (m, 1H), 4.32 (s, 2H), 5.13 (s, 2H), 6.20 (d, 1H), 7.34 (m, 3H), 7.39 (m, 2H), 7.61 (m, 3H), 7.81 (d, 1H), 8.12 (m, 2H), 8.86 (d, 1H), 9.14 (d, 1H).

### Example 44

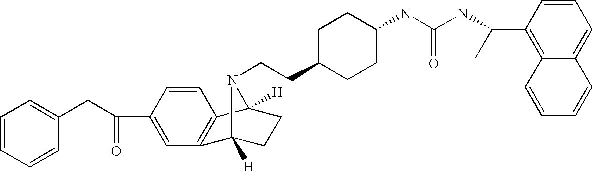

### 1-((S)-1-Naphthalen-1-yl-ethyl)-3-(trans-4-{2-[6-(2-phenyl-ethanoyl)-1,2,3,4-tetrahydro-1,4-epiazano-naphthalen-9-yl]-ethyl}-cyclohexyl)-urea

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and 1-((S)-1-isocyanato-ethyl)-naphthalene by following the procedure in Example 17 (28% yield): LCMS (ES) m/z586 (M+H)⁺; ¹H-NMR(CDCl₃) δ 0.65 (m, 1H), 0.9 (m, 3H), 1.05 (m, 1H), 1.28 (m, 1H), 1.52 (m, 7H), 1.69 (d, 3H), 1.87 (m, 1H), 2.64 (m, 2H), 2.71 (m, 2H), 3.26 (m, 1H), 4.30 (s, 2H), 5.04 (s, 2H), 5.42 (d, 1H), 7.32 (m, 4H), 7.39 (m, 1H), 7.56 (m, 5H), 7.82 (d, 1H), 7.91 (d, 1H), 8.07 (m, 3H).

### Example 45

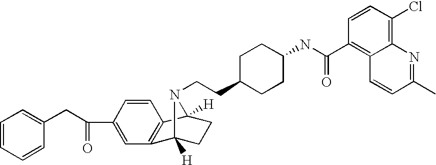

### 8-Chloro-2-methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and quinoline-8-chloro-2-methyl-5-carboxylic acid by following the procedures in Example 19 (26% yield): LCMS (ES) m/z592 (M+H)⁺; ¹H-NMR(CDCl₃) δ1.08 (m, 1H), 1.27 (m, 4H), 1.57 (m, 4H), 1.69 (m, 2H), 2.11 (m, 2H), 2.76 (m, 4H), 2.90 (s, 3H), 3.96 (m, 1H), 4.32 (s, 2H), 5.12 (s, 2H), 6.03 (d, 1H), 7.33 (m, 3H), 7.40 (m, 2H), 7.50 (d, 1H), 7.56 (m, 2H), 7.81 (d, 1H), 8.13 (m, 2H), 8.78 (d, 1H).

### Example 46

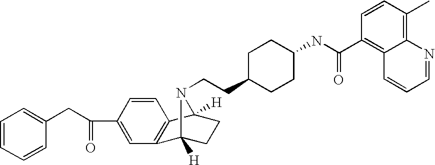

### 8-Methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and quinoline-8-methyl-5-carboxylic acid by following the procedures in Example 19 (93% yield): LCMS (ES) m/z558 (M+H)⁺; ¹H-NMR(CDCl₃) δ 1.05 (m, 1H), 1.23 (m, 4H), 1.56 (m, 4H), 1.73 (m, 2H), 2.11 (m, 2H), 2.71 (m, 2H), 2.77 (m, 2H), 2.94 (s, 3H), 3.96 (m, 1H), 4.32 (s, 2H), 5.12 (s, 2H), 6.31 (d, 1H), 7.33 (m, 3H), 7.40 (m, 2H), 7.55 (d, 1H), 7.80 (m, 2H), 7.89 (m, 1H), 8.13 (m, 2H), 9.40 (d, 1H), 9.43 (d, 1H).

### Example 47

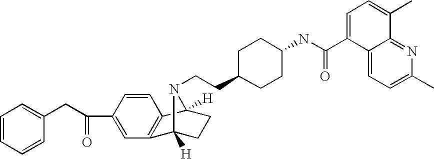

### 2,8-Dimethyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and quinoline-2,8-dimethyl-5-carboxylic acid by following the procedures in Example 19 (59% yield): LCMS (ES) m/z572 (M+H)⁺; ¹H-NMR(CDCl₃) δ 1.05 (m, 1H), 1.23 (m, 4H), 1.57 (m, 4H), 1.73 (m, 2H), 2.10 (m, 2H), 2.73 (m, 4H), 2.93 (s, 3H), 3.08 (s, 3H), 3.94 (m, 1H), 4.32 (s, 2H), 5.00 (s, 2H), 6.28 (d, 1H), 7.30 (m, 3H), 7.38 (m, 2H), 7.55 (d, 1H), 7.65 (d, 1H), 7.74 (m, 2H), 8.10 (m, 2H), 9.32 (d, 1H).

### Example 48

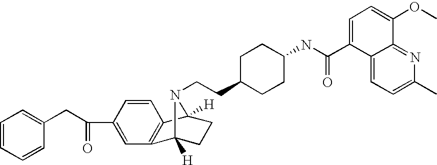

### 8-Methoxy-2-methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and 8-methoxy-2-methyl-quinoline-5-carboxylic acid by following the procedures in Example 19 (33% yield): LCMS (ES) m/z588 (M+H)⁺; ¹H-NMR(CDCl₃) δ1.11 (m, 1H), 1.23 (m, 4H), 1.57 (m, 4H), 1.73 (m, 2H), 2.10 (m, 2H), 2.75 (m, 4H), 3.10 (s, 3H), 3.94 (m, 1H), 4.12 (s, 3H), 4.33 (s, 2H), 5.09 (s, 2H), 6.19 (d, 1H), 7.19 (m, 31H), 7.31 (m, 3H), 7.40 (m, 2H), 7.54 (d, 1H), 7.68 (d, 1H), 7.82 (d, 1H), 8.12 (m, 2H), 9.40 (d, 1H).

### Example 49

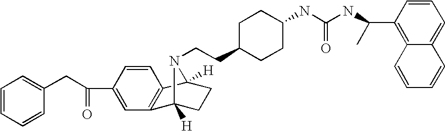

### 1-((R)-1-Naphthalen-1-yl-ethyl)-3-(4-{2-[(1S,4R)-6-(2-phenyl-ethanoyl)-1,2,3,4-tetrahydro-1,4-epiazano-naphthalen-9-yl]-ethyl}-cyclohexyl)-urea

The title compound was prepared from 1-{9-[2-(trans-4-amino-cyclohexyl)-ethyl]-1,2,3,4-tetrahydro-1,4-epiazano-naphthalen-6-yl}-2-phenyl-ethanone and 1-((R)-1-isocyanato-ethyl)-naphthalene by following the procedures in Example 17 (51% yield): LCMS (ES) m/z586 (M+H)⁺; ¹H-NMR(CDCl₃) δ 0.65 (m, 1H), 0.85 (m, 3H), 1.05 (m, 1H), 1.28 (m, 1H), 1.51 (m, 7H), 1.68 (d, 3H), 1.86 (m, 1H), 2.66 (m, 4H), 3.27 (m, 1H), 4.30 (s, 2H), 4.98 (s, 2H), 5.42 (d, 1H), 7.32 (m, 4H), 7.39 (m, 1H), 7.56 (m, 5H), 7.83 (d, 1H), 7.91 (d, 1H), 8.01 (m, 1H), 8.07 (m, 2H).

### Example 50

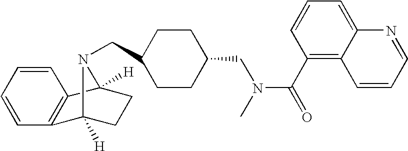

### Quinoline-5-carboxylic acid methyl-{trans-4-[1-(1,2,3,4-tetrahydro-1,4-epiazano-naphthalen-9-yl)methyl]-cyclohexylmethyl}-amide

### 51a) Preparation of methyl-{trans-4-[1-(1,2,3,4-tetrahydro-1,4-epiazano-naphthalen-9-yl)methyl]-cyclohexylmethyl}-amine

To a solution of [trans-4-(1,2,3,4-tetrahydro-1,4-epiazano-naphthalen-9-yl-methanoyl)-cyclohexylmethyl]-carbamic acid tert-butyl ester (0.20 g, 0.52 mmol) in THF (2.0 mL), lithium aluminumhydride (1.0 N in THF, 1.56 mL, 1.56 mmol) was added. The solution was heated with a microwave reactor at 100° C. for 60 minutes before it was mixed with saturated aqueous Na₂SO₄ solution. The resultant mixture was filtered through celite. The organic phases were collected, dried over NaOH, filtered and concentrated to afford the title compound (0.11 g, 77%); LCMS (ES) m/z285 (M+H)⁺; ¹H-NMR(CDCl₃) δ 0.91 (m, 4H), 1.19 (m, 2H), 1.29 (m, 1H), 1.45 (m, 2H), 1.79 (m, 4H), 1.95 (d, 2H), 2.10 (m, 2H), 2.44 (m, 5H), 4.15 (s, 2H), 7.14 (m, 2H), 7.21 (m, 2H).

### 51b) Preparation of quinoline-5-carboxylic acid methyl-{trans-4-[1-(1,2,3,4-tetrahydro-1,4-epiazano-naphthalen-9-yl)methyl]-cyclohexylmethyl}-amide

The title compound was prepared from methyl-{trans-4-[1-(1,2,3,4-tetrahydro-1,4-epiazano-naphthalen-9-yl)methyl]-cyclohexylmethyl}-amine and quinoline-5-carboxylic acid by following the experimental procedure in Example 12d (92% yield): LCMS (ES) m/z440 (M+H)⁺; ¹H-NMR(CDCl₃) δ 0.89 (m, 1H), 1.03 (m, 1H), 1.20 (m, 1H), 1.38 (m, 1H), 1.62 (m, 5H), 1.87 (m, 3H), 2.61 (m, 2H), 2.71 (m, 2H), 2.88 (s, 2H), 3.05 (m, 1H), 3.22 (s, 1H), 3.57 (m, 1H), 4.95 (s, 1H), 5.07 (s, 1H), 7.37 (s, 2H), 7.45 (s, 2H), 7.74 (m, 1H), 7.92 (m, 1H), 8.06 (m, 1H), 8.56 (m, 1H), 8.78 (m, 1H), 9.30 (m, 1H).

### Example 51

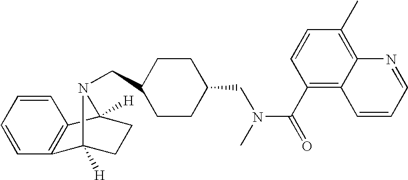

### 8-Methyl-quinoline-5-carboxylic acid methyl-{trans-4-[1-(1,2,3,4-tetrahydro-1,4-epiazano-naphthalen-9-yl)methyl]-cyclohexylmethyl}-amide

The title compound was prepared from methyl-{trans-4-[1-(1,2,3,4-tetrahydro-1,4-epiazano-naphthalen-9-yl)methyl]-cyclohexylmethyl}-amine and 8-methyl-5-quinolinecarboxylic acid by following the experimental procedure in Example 12d (93% yield): LCMS (ES) m/z454 (M+H)⁺; ¹H-NMR(CDCl₃) δ 0.89 (m, 1H), 1.03 (m, 1H), 1.20 (m, 1H), 1.38 (m, 1H), 1.62 (m, 5H), 1.87 (m, 3H), 2.61 (m, 2H), 2.69 (m, 2H), 2.88 (s, 2H), 2.99 (s, 3H), 3.05 (m, 1H), 3.22 (s, 1H), 3.56 (m, 1H), 4.96 (s, 1H), 5.08 (s, 1H), 7.36 (s, 2H), 7.45 (s, 2H), 7.66 (m, 1H), 7.88 (m, 1H), 7.95 (m, 1H), 8.47 (m, 1H), 9.30 (m, 1H).

### Example 52

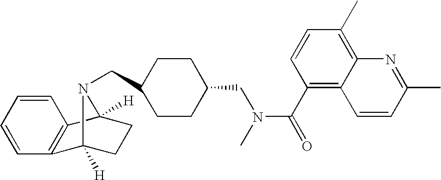

### 2,8-Dimethyl-quinoline-5-carboxylic acid methyl-{trans-4-[1-(1,2,3,4-tetrahydro-1,4-epiazano-naphthalen-9-yl)methyl]-cyclohexylmethyl}-amide

The title compound was prepared from methyl-{trans-4-[1-(1,2,3,4-tetrahydro-1,4-epiazano-naphthalen-9-yl)methyl]-cyclohexylmethyl}-amine and 2,8-dimethyl-5-quinolinecarboxylic acid by following the experimental procedure in Example 12d (74% yield): LCMS (ES) m/z 468(M+H)⁺; ¹H-NMR(CDCl₃) δ 0.88 (m, 1H), 1.02 (m, 1H), 1.19 (m, 1H), 1.38 (m, 1H), 1.61 (m, 5H), 1.85 (m, 3H), 2.59 (m, 2H), 2.68 (m, 2H), 2.86 (s, 1H), 2.95 (s, 3H), 3.13 (s, 3H), 3.07(m, 1H), 3.20 (s, 2H), 3.55 (m, 1H), 4.95 (s, 1H), 5.08 (s, 1H), 7.38 (m, 2H), 7.45 (s, 2H), 7.62 (m, 1H), 7.76 (m, 1H), 7.86 (m, 1H), 8.77 (m, 1H).

### Example 53

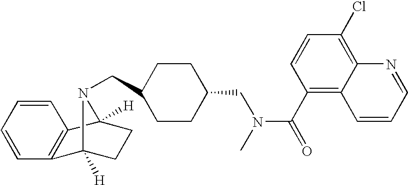

### 8-Chloro-quinoline-5-carboxylic acid methyl-{4-[(1S,4S)-1-1,2,3,4-tetrahydro-1,4-epiazano-naphthalen-9-yl)methyl]-cyclohexylmethyl}-amide

The title compound was prepared from methyl-{trans-4-[1-(1,2,3,4-tetrahydro-1,4-epiazano-naphthalen-9-yl)methyl]-cyclohexylmethyl}-amine and 8-chloro-5-quinolinecarboxylic acid by following the experimental procedure in Example 12d (90% yield): LCMS (ES) m/z 474 (M)⁺; ¹H-NMR(CDCl₃) δ 0.89 (m, 1H), 1.02 (m, 1H), 1.19 (m, 1H), 1.38 (m, 1H), 1.61 (m, 5H), 1.86 (m, 3H), 2.60 (m, 2H), 2.69 (m, 2H), 2.86 (s, 2H), 3.01 (m, 1H), 3.23 (s, 1H), 3.56 (m, 1H), 4.99 (s, 1H), 5.10 (s, 1H), 7.38 (s, 2H), 7.42 (s, 2H), 7.49 (m, 1H), 7.71 (m, 1H), 7.96 (m, 1H), 8.38 (m, 1H), 9.23 (m, 1H).

### Example 54

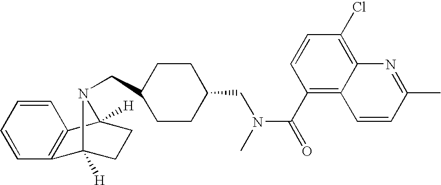

### 8-Chloro-2-methyl-quinoline-5-carboxylic acid methyl-{trans-4-[1-(1,2,3,4-tetrahydro-1,4-epiazano-naphthalen-9-yl)methyl]-cyclohexylmethyl}-amide

The title compound was prepared from methyl-{trans-4-[1-(1,2,3,4-tetrahydro-1,4-epiazano-naphthalen-9-yl)methyl]-cyclohexylmethyl}-amine and 8-chloro-2-methyl-5-quinolinecarboxylic acid by following the experimental procedure in Example 12d (97% yield): LCMS (ES) m/z 488(M)⁺; ¹H-NMR(CDCl₃) δ 0.87 (m, 1H), 1.01 (m, 1H), 1.18 (m, 1H), 1.38 (m, 1H), 1.61 (m, 5H), 1.85 (m, 3H), 2.59 (m, 2H), 2.68 (m, 2H), 2.86 (s, 1H), 3.02 (s, 3H), 3.08(m, 1H), 3.22 (s, 2H), 3.55 (m, 1H), 4.97 (s, 1H), 5.09 (s, 1H), 7.38 (m, 2H), 7.46 (s, 2H), 7.53 (m, 1H), 7.68 (m, 1H), 7.99 (m, 1H), 8.47 (m, 1H).

In addition, following either the procedure for the preparation of Example 17 (ureas) or for the preparation of Example 12 (amides), the following compounds (Examples 56-136) were synthesized and tested:

TABLE 1

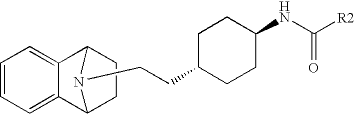

| | | LC/MS |
|---|---|---|
| | | (ES) |
| Example | Compound Name | (M + H)⁺ |
| 55 | 1-(trans-4-[2-(1,2,3,4-tetrahydro-1,4-epiazano- | 390.0 |
| | naphthalen-9-yl)-ethyl]-cyclohexyl)-2-(4- | |
| | pyridinyl)acetamide | |
| 56 | 1-(trans-4-[2-(1,2,3,4-tetrahydro-1,4-epiazano- | 405.0 |
| | naphthalen-9-yl)-ethyl]-cyclohexyl)-3-(2- | |
| | pyridinylmethyl)urea | |
| 57 | 1-(trans-4-[2-(1,2,3,4-tetrahydro-1,4-epiazano- | 410.0 |
| | naphthalen-9-yl)-ethyl]-cyclohexyl)-3- | |
| | (cyclohexylmethyl)urea | |
| 58 | 1-(trans-4-[2-(1,2,3,4-tetrahydro-1,4-epiazano- | 412.0 |
| | naphthalen-9-yl)-ethyl]-cyclohexyl)-3-(4- | |
| | hydroxycyclohexyl)urea | |
| 59 | N′-(trans-4-[2-(1,2,3,4-tetrahydro-1,4-epiazano- | 418.0 |
| | naphthalen-9-yl)-ethyl]-cyclohexyl)-N-methyl-N- | |
| | (phenylmethyl)urea | |
| 60 | 1-(trans-4-[2-(1,2,3,4-tetrahydro-1,4-epiazano- | 419.0 |
| | naphthalen-9-yl)-ethyl]-cyclohexyl)-3-[2-(2- | |
| | pyridinyl)ethyl]urea | |
| 61 | 1-(trans-4-[2-(1,2,3,4-tetrahydro-1,4-epiazano- | 420.0 |
| | naphthalen-9-yl)-ethyl]-cyclohexyl)-3-[2- | |
| | hydroxyphenyl) methyl]urea | |
| 62 | 1-(trans-4-[2-(1,2,3,4-tetrahydro-1,4-epiazano- | 421.0 |
| | naphthalen-9-yl)-ethyl]-cyclohexyl)-3-[2-(1- | |
| | methyl-1H-pyrrol-2-yl)ethyl]urea | |
| 63 | 1-(trans-4-[2-(1,2,3,4-tetrahydro-1,4-epiazano- | 422.0 |
| | naphthalen-9-yl)-ethyl]-cyclohexyl)-3-[4- | |
| | fluorophenyl)methyl]urea | |
| 64 | N-(trans-4-[2-(1,2,3,4-tetrahydro-1,4-epiazano- | 423.0 |
| | naphthalen-9-yl)-ethyl]-cyclohexyl)-2-(2- | |
| | pyrimidinylthio)aoetamide | |
| 65 | N-(trans-4-[2-(1,2,3,4-tetrahydro-1,4-epiazano- | 426.0 |
| | naphthalen-9-yl)-ethyl]-cyclohexyl)-3- | |
| | quinolinecarboxamide | |
| 66 | N-(trans-4-[2-(l,2,3,4-tetrahydro-1,4-epiazano- | 428.0 |
| | naphthalen-9-yl)-ethyl]-cyclohexyl)-1-methyl-1H- | |
| | indole-2-carboxamide | |
| 67 | (2E)-N-(trans-4-[2-(1,2,3,4-tetrahydro-1,4- | 429.0 |
| | epiazano-naphthalen-9-yl)-ethyl]-Cyclohexyl)-4- | |
| | oxo-4-phenyl-2-butenamide | |
| 68 | 1-(trans-4-[2-(1,2,3,4-tetrahydro-1,4-epiazano- | 430.0 |
| | naphthalen-9-yl)-ethyl]-cyclohexyl)-3-(2,3- | |
| | dihydro-1 H-inden-1-yl)urea | |
| 69 | 1-(trans-4-[2-(1,2,3,4-tetrahydro-1,4-epiazano- | 432.0 |
| | naphthalen-9-yl)-ethyl]-cyclohexyl)-3-(3- | |
| | phenylpropyl)urea | |
| 70 | 1-(trans-4-[2-(1,2,3,4-tetrahydro-1,4-epiazano- | 439.0 |
| | naphthalen-9-yl)-ethyl]-cyclohexyl)-3-t(4- | |
| | ohlorophenyl)methyl]urea | |
| 71 | 1-(trans-4-[2-(1,2,3,4-tetrahydro-1,4-epiazano- | 442.0 |
| | naphthalen-9-yl)-ethyl]-cyclohexyl)-3-(1,1a,6,6a- | |
| | tetrahydrocyclopropata]inden-1-yl) urea | |
| 72 | 1-(trans-4-[2-(1,2,3,4-tetrahydro-1,4-epiazano- | 443.0 |
| | naphthalen-9-yl)-ethyl]-cyclohexyl)-3-(1H-indol- | |
| | 3-ylmethyl)urea | |
| 73 | 1-(trans-4-[2-(1,2,3,4-tetrahydro-1,4-epiazano- | 444.0 |
| | naphthalen-9-yl)-ethyl]-cyclohexyl)-3-(1H- | |
| | benzim idazol-2-ylmethyl)urea | |
| 74 | 1-(trans-4-[2-(1,2,3,4-tetrahydro-1,4-epiazano- | 444.0 |
| | naphthalen-9-yl)-ethyl]-cyclohexyl)-3-(1,2,3,4- | |
| | tetrahydro-2-naphthalenyl)urea | |
| 75 | 1-(trans-4-[2-(1,2,3,4-tetrahydro-1,4-epiazano- | 444.0 |
| | naphthalen-9-yl)-ethyl]-cyclohexyl)-3-(1,2,3,4- | |
| | tetrahydro-1-naphthalenyl)urea | |
| 76 | 1-(trans-4-[2-(1,2,3,4-tetrahydro-1,4-epiazano- | 446.0 |
| | naphthalen-9-yl)-ethyl]-cyclohexyl)-N,N- | |
| | dimethyiphenylalaninamide | |
| 77 | 1-(trans-4-[2-(1,2,3,4-tetrahydro-1,4-epiazano- | 446.0 |
| | naphthalen-9-yl)-ethyl]-cyclohexyl)-3-(4- | |
| | phenylbutyl)urea | |
| 78 | 1-(trans-4-[2-(1,2,3,4-tetrahydro-1,4-epiazano- | 458.0 |
| | naphthalen-9-yl)-ethyl]-cyclohexyl)-3-(2-methyl- | |
| | 1,2,3,4-tetrahydro-2-naphthalenyl)urea | |
| 79 | N-(trans-4-[2-(1,2,3,4-tetrahydro-1,4-epiazano- | 460.0 |
| | naphthalen-9-yl)-ethyl]-cyclohexyl)-4-(2- | |
| | pyridinyl)-1-piperazinecarboxamide | |
| 80 | N′-(trans-4-[2-(1,2,3,4-tetrahydro-1,4-epiazano- | 462.0 |
| | naphthalen-9-yl)-ethyl]-cyclohexyl)-N-(3- | |
| | hydroxypropyl)-N-(phenylmethyl)urea | |
| 81 | 1-(trans-4-[2-(1,2,3,4-tetrahydro-1,4-epiazano- | 419.0 |
| | naphthalen-9-yl)-ethyl]-cyclohexyl)-3-[2-(4- | |
| | pyridinyl)ethyl]urea formate | |
| 82 | N-(trans-4-[2-(1,2,3,4-tetrahydro-1,4-epiazano- | 465.0 |
| | naphthalen-9-yl)-ethyl]-cyclohexyl)-2,2- | |
| | diphenylacetamide | |
| 83 | 1-(trans-4-[2-(1,2,3,4-tetrahydro-1,4-epiazano- | 422.0 |
| | naphthalen-9-yl)-ethyl]-cyclohexyl)-3-[3-(1 H- | |
| | imidazol-1-yl)propyl]urea formate | |
| 84 | 1-(trans-4-[2-(1,2,3,4-tetrahydro-1,4-epiazano- | 472.0 |
| | naphthalen-9-yl)-ethyl]-cyclohexyl)-3-{[4- | |
| | (trifluoromethyl)phenyl]methyl}urea | |
| 85 | N-(trans-4-[2-(1,2,3,4-tetrahydro-1,4-epiazano- | 473.0 |
| | naphthalen-9-yl)-ethyl]-cyclohexyl)-4- | |
| | (phenylmethyl)-1-piperazinecarboxamide | |
| 86 | N-{5-[(trans-4-[2-(1,2,3,4-tetrahydro-1,4- | 474.0 |
| | epiazano-naphthalen-9-yl)-ethyl]- | |
| | cyclohexyl)amino]-5-oxopentyl}benzamide | |
| 87 | 1,1-dimethylethyl 2-{[(trans-4-[2-(1,2,3,4- | 475.0 |
| | tetrahydro-1,4-epiazano-naphthalen-9-yl)-ethyl]- | |
| | cyclohexyl)amino]carbonyl}benzoate | |
| 88 | N-(trans-4-[2-(1,2,3,4-tetrahydro-1,4-epiazano- | 479.0 |
| | naphthalen-9-yl)-ethyl]-cyclohexyl)-2,2- | |
| | diphenyipropanamide | |
| 89 | N-(trans-4-[2-(1,2,3,4-tetrahydro-1,4-epiazano- | 479.0 |
| | naphthalen-9-yl)-ethyl]-cyclohexyl)-3,3- | |
| | diphenyipropanamide | |
| 90 | 1-(trans-4-[2-(1,2,3,4-tetrahydro-1,4-epiazano- | 443.0 |
| | naphthalen-9-yl)-ethyl]-cyclohexyl)-3-(1H-indol- | |
| | 3-ylmethyl)urea formate | |
| 91 | 1-(trans-4-[2-(1,2,3,4-tetrahydro-1,4-epiazano- | 447.0 |
| | naphthalen-9-yl)-ethyl]-cyclohexyl)-3-{[3- | |
| | (dimethylamino)phenyl]methyl}urea formate | |
| 92 | N-(trans-4-[2-(1,2,3,4-tetrahydro-1,4-epiazano- | 493.0 |
| | naphthalen-9-yl)-ethyl]-cyclohexyl)-3-(4- | |
| | methylphenyl)-3-phenylpropanamide | |
| 93 | N-(trans-4-[2-(1,2,3,4-tetrahydro-1,4-epiazano- | 493.0 |
| | naphthalen-9-yl)-ethyl]-cyclohexyl)-4,4- | |
| | diphenylbutanamide | |
| 94 | N-(trans-4-[2-(1,2,3,4-tetrahydro-1,4-epiazano- | 494.0 |
| | naphthalen-9-yl)-ethyl]-cyclohexyl)-4- | |
| | [(phenylcarbonyl)amino]benzamide | |
| 95 | 1-(trans-4-[2-(1,2,3,4-tetrahydro-1,4-epiazano- | 494.0 |
| | naphthalen-9-yl)-ethyl]-cyclohexyl)-3-(1,2- | |
| | diphenylethyl)urea | |
| 96 | 1-(trans-4-[2-(1,2,3,4-tetrahydro-1,4-epiazano- | 494.0 |
| | naphthalen-9-yl)-ethyl]-cyclohexyl)-3-(2,2- | |
| | diphenylethyl)urea | |
| 97 | 1-(trans-4-[2-(1,2,3,4-tetrahydro-1,4-epiazano- | 494.0 |
| | naphthalen-9-yl)-ethyl]-cyclohexyl)-3-(2,2- | |
| | diphenylethyl)urea | |
| 98 | N′-(trans-4-[2-(1,2,3,4-tetrahydro-1,4-epiazano- | 494.0 |
| | naphthalen-9-yl)-ethyl]-cyclohexyl)-N,1- | |
| | bis(phenylmethyl)urea | |
| 99 | 1-(trans-4-[2-(1,2,3,4-tetrahydro-1,4-epiazano- | 495.0 |
| | naphthalen-9-yl)-ethyl]-cyclohexyl)-2- | |
| | (methyloxy)-2,2-diphenylacetamide | |
| 100 | 1-(trans-4-[2-(1,2,3,4-tetrahydro-1,4-epiazano- | 454.0 |
| | naphthalen-9-yl)-ethyl]-cyclohexyl)-3-(1- | |
| | naphthalenylmethyl)urea formate | |
| 101 | 1-(trans-4-[2-(1,2,3,4-tetrahydro-1,4-epiazano- | 508.0 |
| | naphthalen-9-yl)-ethyl]-cyclohexyl)-3-(3,3- | |
| | diphenylpropyl)urea | |
| 102 | N′-(trans-4-[2-(1,2,3,4-tetrahydro-1,4-epiazano- | 508.0 |
| | naphthalen-9-yl)-ethyl]-cyclohexyi)-1-(2- | |
| | phenylethyl)-1-(phenylmethyl)urea | |
| 103 | 1-(trans-4-[2-(1,2,3,4-tetrahydro-1,4-epiazano- | 510.0 |
| | naphthalen-9-yl)-ethyl]-cyclohexyl)-3-(2- | |
| | hydroxy-2,2-diphenylethyl)urea | |
| 104 | N-[2-({[(trans-4-[2-(1,2,3,4-tetrahydro-1,4- | 511.0 |
| | epiazano-naphthalen-9-yl)-ethyl]- | |
| | cyclohexyl)amino]carbonyl}amino)ethyl]-4- | |
| | methylbenzenesulfonamide | |
| 105 | 1,1-dimethylethyl N-{[(trans-4-[2-(1,2,3,4- | 518.0 |
| | tetrahydro-1,4-epiazano-naphthalen-9-yl)-ethyl]- | |
| | cyclohexyl)amino]carbonyl}-L-phenylalaninate | |
| 106 | N-(trans-4-[2-(1,2,3,4-tetrahydro-1,4-epiazano- | 473.0 |
| | naphthalen-9-yl)-ethyl]-cyclohexyl)-4- | |
| | (phenylmethyl)-1-piperazinecarboxamide | |
| | formate | |
| 107 | N′-(trans-4-[2-(1,2,3,4-tetrahydro-1,4-epiazano- | 522.0 |
| | naphthalen-9-yl)-ethyl]-cyclohexyl)-N-(3,3- | |
| | diphenylpropyl)-N-methylurea | |
| 108 | 1-(trans-4-[2-(1,2,3,4-tetrahydro-1,4-epiazano- | 525.0 |
| | naphthalen-9-yl)-ethyl]-cyclohexyl)-3-(2-{3- | |
| | [hydroxy(3-pyridinyl)methyl]phenyl}ethyl)urea | |
| 109 | 3-[({[(trans-4-[2-(1,2,3,4-tetrahydro-1,4- | 483.0 |
| | epiazano-naphthalen-9-yl)-ethyl]- | |
| | cyclohexyl)aminolcarbonyl}amino) | |
| | methyl]benzene-sulfonamide formate | |
| 110 | 1-(trans-4-[2-(1,2,3,4-tetrahydro-1,4-epiazano- | 360.0 |
| | naphthalen-9-yl)-ethyl]-cyclohexyl)-3-[1- | |
| | (phenylmethyl)-4-piperidinyl]urea formate | |
| 111 | 1-(trans-4-[2-(1,2,3,4-tetrahydro-1,4-epiazano- | 432.0 |
| | naphthalen-9-yl)-ethyl]-cyclohexyl)-3-(3- | |
| | phenylpropyl)urea trifluoroacetate | |
| 112 | 1-(trans-4-[2-(1,2,3,4-tetrahydro-1,4-epiazano- | 504.0 |
| | naphthalen-9-yl)-ethyl}-cyclohexyl)-3-[5,8- | |
| | bis(methyloxy)-1,2,3,4-tetrahydro-2- | |
| | naphthalenyl]urea formate | |
| 113 | N′-(trans-4-[2-(1,2,3,4-tetrahydro-1,4-epiazano- | 550.0 |
| | naphthalen-9-yl)-ethyl]-cyclohexyl)-N-(3,3- | |
| | diphenylpropyl)-N-propylurea | |
| 114 | 1-(trans-4-[2-(1,2,3,4-tetrahydro-1,4-epiazano- | 508.0 |
| | naphthalen-9-yl)-ethyl]-cyclohexyl)-3-(3,3- | |
| | diphenylpropyl)urea formate | |
| 115 | 1-(trans-4-[2-(1,2,3,4-tetrahydro-1,4-epiazano- | 508.0 |
| | naphthalen-9-yl)-ethyl]-cyclohexyl)-3-(1-methyl- | |
| | 2,2-diphenylethyl)urea formate | |
| 116 | N-(trans-4-[2-(1,2,3,4-tetrahydro-1,4-epiazano- | 555.0 |
| | naphthalen-9-yl)-ethyl]-cyclohexyl)-2-[(2- | |
| | methylphenyl)(phenyl)methyl]benzamide | |
| 117 | 1-(trans-4-[2-(1,2,3,4-tetrahydro-1,4-epiazano- | 510.0 |
| | naphthalen-9-yl)-ethyl]-cyclohexyl)-3-(2- | |
| | hydroxy-1,1-diphenylethyl)urea formate | |
| 118 | N-(trans-4-[2-(1,2,3,4-tetrahydro-1,4-epiazano- | 564.0 |
| | naphthalen-9-yl)-ethyl]-cyclohexyl)-4- | |
| | (diethylamino)-2,2-diphenylbutanamide | |
| 119 | N,N′-bis(trans-4-[2-(1,2,3,4-tetrahydro-1,4- | 567.0 |
| | epiazano-naphthalen-9-yl)-ethyl]- | |
| | cyclohexyl)urea | |
| 120 | 1-(trans-4-[2-(1,2,3,4-tetrahydro-1,4-epiazano- | 524.0 |
| | naphthalen-9-yl)-ethyl]-cyclohexyl)-3-(3- | |
| | hydroxy-3,3-diphenylpropyl)urea formate | |
| 121 | 1-(trans-4-[2-(1,2,3,4-tetrahydro-1,4-epiazano- | 524.0 |
| | naphthalen-9-yl)-ethyl]-cyclohexyl)-3-[(1S)-2- | |
| | hydroxy-1-methyl-2,2-diphenylethyl]urea | |
| | formate | |
| 122 | 1-(trans-4-[2-(1,2,3,4-tetrahydro-1,4-epiazano- | 525.0 |
| | naphthalen-9-yl)-ethyl]-cyclohexyl)-3-(2-{3- | |
| | [hydroxy(3-pyridinyl)methyl]phenyl}ethyl)urea | |
| | formate | |
| 123 | 1-(trans-4-[2-(1,2,3,4-tetrahydro-1,4-epiazano- | 536.0 |
| | naphthalen-9-yl)-ethyl]-cyclohexyl)-3-(1,1- | |
| | dimethyl-3,3-diphenylpropyl)urea formate | |
| 124 | N′-(trans-442-(1,2,3,4-tetrahydro-1,4-epiazano- | 536.0 |
| | naphthalen-9-yl)-ethyl]-cyclohexyl)-N-(3,3- | |
| | diphenylpropyl)-N-ethylurea formate | |
| 125 | N′-(trans-4-[2-(1,2,3,4-tetrahydro-1,4-epiazano- | 584.0 |
| | naphthalen-9-yl)-ethyl]-cyclohexyl)-N-methyl-N- | |
| | (2,2,2-triphenylethyl)urea | |
| 126 | N-(trans-4-[2-(1,2,3,4-tetrahydro-1,4-epiazano- | 585.0 |
| | naphthalen-9-yl)-ethyl]-cyclohexyl)-3-phenyl-3- | |
| | {3-[(phenylmethyl)oxy]phenyl}propanamide | |
| 127 | N-(trans-4-[2-(1,2,3,4-tetrahydro-1,4-epiazano- | 481.0 |
| | naphthalen-9-yl)-ethyl]-cyclohexyl)-2-hydroxy- | |
| | 2,2-diphenylacetamide trifluoroacetate (salt) | |
| 128 | N′-(trans-4-[2-(1,2,3,4-tetrahydro-1,4-epiazano- | 552.0 |
| | naphthalen-9-yl)-ethyl]-cyclohexyl)-N-ethyl-N-(3- | |
| | hydroxy-3,3-diphenylpropyl)urea formate | |
| 129 | N-(trans-4-[2-(1,2,3,4-tetrahydro-1,4-epiazano- | 314.0 |
| | naphthalen-9-yl)-ethyl]-cyclohexyl)-2-{bis[4- | |
| | (dimethylamino)phenyl]methyl}benzamide | |
| 130 | N-(trans-4-[2-(1,2,3,4-tetrahydro-1,4-epiazano- | 522.0 |
| | naphthalen-9-yl)-ethyl]-cyclohexyl)-3-[4- | |
| | (dimethylamino)phenyl]-3-phenylpropanamide | |
| | trifluoroacetate | |
| 131 | N′-(trans-4-[2-(1,2,3,4-tetrahydro-1,4-epiazano- | 598.0 |
| | naphthalen-9-yl)-ethyl]-cyclohexyl)-N-(3,3 | |
| | diphenylpropyl)-N-(Phenylmethyl)urea formate | |
| 132 | N-(trans-4-[2-(1,2,3,4-tetrahydro-1,4-epiazano- | 533.0 |
| | naphthalen-9-yl)-ethyl]-cyclohexyl)-2,2-bis(4- | |
| | chlorophenyl)acetamide trifluoroacetate | |
| 133 | N-(trans-4-[2-(1,2,3,4-tetrahydro-1,4-epiazano- | 564.0 |
| | naphthalen-9-yl)-ethyl]-cyclohexyl)-4- | |
| | (diethylamino)-2,2-dipheflylbutanamide | |
| | trifluoroacetate | |
| 134 | 1-(trans-4-[2-(1,2,3,4-tetrahydro-1,4-epiazano- | 634.0 |
| | naphthalen-9-yl)-ethyl]-Cyclohexyl)-3-[3(4 | |
| | biphenylyl)-3-(4-chlorophenyl)-3- | |
| | hydroxypropyl]urea formate | |
| 135 | N′-(trans-4-[2-(1,2,3,4-tetrahydro-1,4-epiazano- | 584.0 |
| | naphthalen-9-yl)-ethyl]-cyclohexyl)-N-(2,2- | |
| | diphenylethyl)-N-(phenylmethyl)urea | |
| | trifluoroacetate | |

### BIOLOGICAL EXAMPLES

The inhibitory effects of compounds at the M₃ mAChR of the present invention are determined by the following in vitro and in vivo functional assays:

Analysis of Inhibition of Receptor Activation by Calcium Mobilization:

Stimulation of mAChRs expressed on CHO cells were analyzed by monitoring receptor-activated calcium mobilization as previously described (Sarau, H. M., R. S. Ames, J. Chambers, C. Ellis, N. Elshourbagy, J. J. Foley, D. B. Schmidt R. M. Muccitelli, O. Jenkins, P. R. Murdock, N.C. Herrity, W. Halsey, G. Sathe, A. I. Muir, P. Nuthulaganti, G. M. Dytko, P. T. Buckley, S. Wilson, D. J., Bergsma, and D. W. Hay. 1999. Identification, molecular cloning, expression, and characterization of a cysteinyl leukotriene receptor. Mol Pharmacol 56:657-663). CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels (Sullivan, E., E. M. Tucker, and 1. L. Dale. 1999. Measurement of [Ca2+] using the Fluorometric Imaging Plate Reader (FLIPR). Methods Mol Biol 114:125-133). The emitted fluorescence from all 96 wells is measured simultaneously using a cooled CCD camera. Data points are collected every second. This data was then plotting and analyzed using GraphPad PRISM software.

Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine (Hamelmann, E., J. SCHWARZE, K. TAKEDA, A. OSHIBA, G. á. LARSEN, C. á. IRVIN, and E. á. GELFAND. 1997. Noninvasive Measurement of Airway Responsiveness in Allergic Mice Using Barometric Plethysmography. Am. J. Respir. Crit. Care Med. 156:766-775). Mice were pretreated with 50 μl of compound (0.003-10 μg/mouse) in 50 μl of vehicle (10% DMSO) intranasally, i.v., i.p. or p.o, and were then placed in the plethysmography chamber. Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis; gastrointestinal-tract disorders such as irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakisuria, neurogenic bladder, nocturnal enuresis, psychosomatic bladder, incontinence associated with bladder spasms or chronic cystitis, urinary urgency or pollakiuria, and motion sickness.

Methods of administering the present compounds will be readily apparent to the skilled artisan.

Dry powder compositions for topical delivery to the lung by inhalation may, for example, be presented in capsules and cartridges of for example gelatine, or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 20 μg-10 mg of the compound of formula (I) optionally in combination with another therapeutically active ingredient. Alternatively, the compound of the invention may be presented without excipients.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognised as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

A preferable means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomisation of the spray is ensured as it will not release the formulation until the threshold pressure for effectively atomising the spray has been achieved. Typically, the VP7 model may be used with a bottle capable of holding 10-50 ml of a formulation. Each spray will typically deliver 50-100 μl of such a formulation, therefore, the VP7 model is capable of providing at least 100 metered doses.

### EXAMPLES OF NASAL FORMULATIONS

### Example 1

### Nasal Formulation Containing Active

A formulation for intranasal delivery was prepared with ingredients as follows:

| | | |
|---|---|---|
| | to 100% | |
| Active | 0.1% | w/w |
| Polysorbate 80 | 0.025% | w/w |
| Avicel RC591 | 1.5% | w/w |
| Dextrose | 5.0% | w/w |
| BKC | 0.015% | w/w |
| EDTA | 0.015% | w/w |
| water | to 100% | |

in a total amount suitable for 120 actuations and the formulation was filled into a bottle fitted with a metering valve adapted to dispense 50 or 100 μl per actuation. The device was fitted into a nasal actuator (Valois).

### Example 2

### Nasal Formulation Containing Active

A formulation for intranasal delivery was prepared with ingredients as follows:

| | | |
|---|---|---|
| Active | 0.005% | w/w |
| Tyloxapol | 2% | w/w |
| dextrose | 5% | w/w |
| BKC | 0.015% | w/w |
| EDTA | 0.015% | w/w |
| water | to 100% | |

in a total amount suitable for 120 actuations and the formulation was filled into a bottle (plastic or glass) fitted with a metering valve adapted to dispense 50 or 100 μl per actuation
The device was fitted into a nasal actuator (Valois, e.g. VP3, VP7 or VP7D)

### Example 3

### Nasal Formulation Containing Active

A formulation for intranasal delivery was prepared with ingredients as follows:

| | | |
|---|---|---|
| active | 0.05% | w/w |
| Triton X-100 | 5% | w/w |
| Dextrose | 4% | w/w |
| BKC | 0.015% | w/w |
| EDTA | 0.015% | w/w |
| water | to 100% | |

in a total amount suitable for 120 actuations and the formulation was filled into a bottle fitted with a metering valve adapted to dispense 50 or 100 μl per actuation.

### Example 4

### Nasal Formulation Containing Active

A formulation for intranasal delivery was prepared with ingredients as follows:

| | | |
|---|---|---|
| active | 0.05% | w/w |
| Tyloxapol | 5% | w/w |
| dextrose | 5% | w/w |
| BKC | 0.015% | w/w |
| EDTA | 0.015% | w/w |
| water | to 100% | |

in a total amount suitable for 120 actuations and the formulation was filled into a bottle fitted with a metering valve adapted to dispense 50 or 100 μl per actuation The device was fitted into a nasal actuator (Valois).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

The patents and patent applications described in this application are herein incorporated by reference.

What is claimed is:

1. A compound according to Formula I herein below: 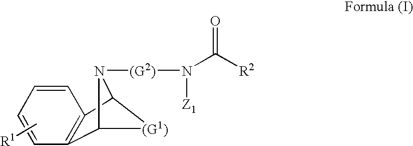
wherein:
Z1 is, independently, selected from the group consisting of H or C₁₋₆ alkyl;
R¹ is, independently, selected from the group consisting of a substituent selected from: Hydrogen, halogen, C₁₋₄ alkyl, —C(O)(C₁₋₆ alkyl), —CO₂(C₁₋₆ alkyl), —C(O)(aryl) and —C(O)[(C₁₋₆ alkyl)-aryl];
G¹ is, independently, selected from the group consisting of CH₂—CH₂ or CH═CH;
G² is, independently, selected from the group consisting of C₄₋₇alkyl or a group of the formula (a), (b) or (c): 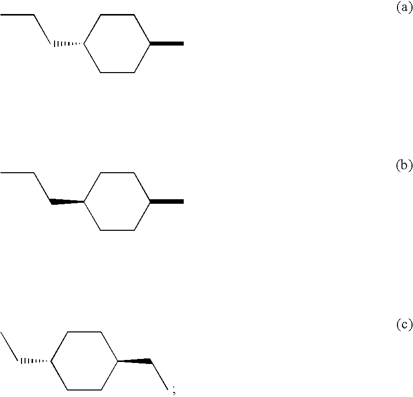
R² is, independently, selected from the group consisting of the formula (d) or (e): 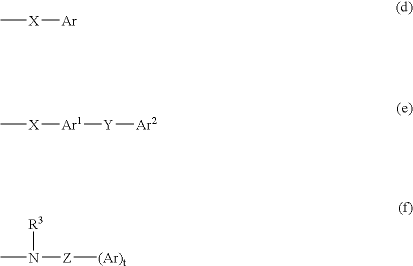
wherein
X is, independently, selected from the group consisting of a bond, NR³ or C₁₋₄ alkyl;
R³ is, independently, selected from the group consisting of H, optionally substituted C₁₋₆ alkyl and C₁₋₄ alkyl-aryl;
Z is, independently, selected from the group consisting of optionally substituted C₁₋₆ alkyl, C₁₋₆ alkyl-Y²; In addition, Z and R³ or Z and Ar may come together to form a 4-7 membered ring;
Ar is, independently, selected from the group consisting of an optionally substituted phenyl ring or an optionally substituted 5- or 6-membered aromatic heterocyclic ring; or an optionally substituted bicyclic or heterobicyclic ring system; or an optionally substituted tricyclic or heterotricyclic ring system;
Ar¹ and Ar² are, independently, selected from a group consisting of an optionally substituted phenyl ring or an optionally substituted 5- or 6-membered aromatic heterocyclic ring;
Y is, independently, selected from a group consisting of a bond, —NHCO—, —CONH—, —CH₂—, and —(CH₂)ₘY¹(CH₂)ₙ— wherein Y¹ represents O, S, SO₂, or CO and m and n each represent zero or 1 such that the sum of m+n is zero or 1; provided that when R² represents a group of formula (d) wherein X is a bond, any substituent present in Ar ortho to the carboxamide moiety is necessarily a hydrogen or a methoxy group
Y² is, independently, selected from a group consisting of NR³, O, S, —NHC(O)—, —C(O)NH—;
t is, independently, selected from a group consisting of an integer between 0 and 3.

2. A compound according to claim 1 consisting of the group selected from:
2-Methyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
8-Chloro-2-methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
8-Methyl-quinoline-5-carboxylic acid (4-{2-[6-butyryl-1,2,3,4-tetrahydro-1,4-epiazano-naphthalen-9-yl]-ethyl}-cyclohexyl)-amide;
2-Methyl-quinoline-5-carboxylic acid (trans-4-(2-[6-butyryl-1,2,3,4-tetrahydro-1,4-epiazano-naphthalen-9-yl]-ethyl}-cyclohexyl)-amide;
2-Methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
N′-(trans-4-[2-(1,2,3,4-tetrahydro-1,4-epiazano-naphthalen-9-yl)-ethyl]-cyclohexyl)-1-(2-phenylethyl)-1-(phenylmethyl)urea;
1-(trans-4-[2-(1,2,3,4-tetrahydro-1,4-epiazano-naphthalen-9-yl)-ethyl]-cyclohexyl)-3-(2-hydroxy-2,2-diphenylethyl)urea;
N-[2-({[(trans-4-[2-(1,2,3,4-tetrahydro-1,4-epiazano-naphthalen-9-yl)-ethyl]-cyclohexyl)amino]carbonyl}amino)ethyl]-4-methylbenzenesulfonamide;
1,1-dimethylethyl N-{[(trans-4-[2-(1,2,3,4-tetrahydro-1,4-epiazano-naphthalen-9-yl)-ethyl]-cyclohexyl)amino]carbonyl}-L-phenylalaninate;
N′-(trans-4-[2-(1,2,3,4-tetrahydro-1,4-epiazano-naphthalen-9-yl)-ethyl]-cyclohexyl)-N-(3,3-diphenylpropyl)-N-methylurea;
3-[({[(trans-4-[2-(1,2,3,4-tetrahydro-1,4-epiazano-naphthalen-9-yl)-ethyl]-cyclohexyl)amino]carbonyl}amino)methyl]benzenesulfonamide formate;
1-(trans-4-[2-(1,2,3,4-tetrahydro-1,4-epiazano-naphthalen-9-yl)-ethyl]-cyclohexyl)-3-(2-hydroxy-1,1-diphenylethyl)urea formate;
N,N′-bis(trans-4-[2-(1,2,3,4-tetrahydro-1,4-epiazano-naphthalen-9-yl)-ethyl]-cyclohexyl)urea
1-(trans-4-[2-(1,2,3,4-tetrahydro-1,4-epiazano-naphthalen-9-yl)-ethyl]-cyclohexyl)-3-(3-hydroxy-3,3-diphenylpropyl)urea formate;
1-(trans-4-[2-(1,2,3,4-tetrahydro-1,4-epiazano-naphthalen-9-yl)-ethyl]-cyclohexyl)-3-[(1S)-2-hydroxy-1-methyl-2,2-diphenylethyl]urea formate;
1-(trans-4-[2-(1,2,3,4-tetrahydro-1,4-epiazano-naphthalen-9-yl)-ethyl]-cyclohexyl)-3-(cyclohexylmethyl)urea;
1-(trans-4-[2-(1,2,3,4-tetrahydro-1,4-epiazano-naphthalen-9-yl)-ethyl]-cyclohexyl)-3-[(2-hydroxyphenyl)methyl]urea;
1-(trans-4-[2-(1,2,3,4-tetrahydro-1,4-epiazano-naphthalen-9-yl)-ethyl]-cyclohexyl)-3-[2-(1-methyl-1H-pyrrol-2-yl)ethyl]urea;
1-(trans-4-[2-(1,2,3,4-tetrahydro-1,4-epiazano-naphthalen-9-yl)-ethyl]-cyclohexyl)-3-[(4-fluorophenyl)methyl]urea;
1-(trans-4-[2-(1,2,3,4-tetrahydro-1,4-epiazano-naphthalen-9-yl)-ethyl]-cyclohexyl)-3-[(4-fluorophenyl)methyl]urea;
1-(trans-4-[2-(1,2,3,4-tetrahydro-1,4-epiazano-naphthalen-9-yl)-ethyl]-cyclohexyl)-3-(2,3-dihydro-1H-inden-1-yl)urea;
1-(trans-4-[2-(1,2,3,4-tetrahydro-1,4-epiazano-naphthalen-9-yl)-ethyl]-cyclohexyl)-3-(3-phenylpropyl)urea;
1-(trans-4-[2-(1,2,3,4-tetrahydro-1,4-epiazano-naphthalen-9-yl)-ethyl]-cyclohexyl)-3-[(4-chlorophenyl)methyl]urea;
1-(trans-4-[2-(1,2,3,4-tetrahydro-1,4-epiazano-naphthalen-9-yl)-ethyl]-cyclohexyl)-3-(1,1a,6,6a-tetrahydrocyclopropa[a]inden-1-yl)urea;
N′-(trans-4-[2-(1,2,3,4-tetrahydro-1,4-epiazano-naphthalen-9-yl)-ethyl]-cyclohexyl)-N-(3-hydroxypropyl)-N-(phenylmethyl)urea;
1-(trans-4-[2-(1,2,3,4-tetrahydro-1,4-epiazano-naphthalen-9-yl)-ethyl]-cyclohexyl)-3-{[4-(trifluoromethyl)phenyl]methyl}urea;
1,1-dimethylethyl 2-{[(trans-4-[2-(1,2,3,4-tetrahydro-1,4-epiazano-naphthalen-9-yl)-ethyl]-cyclohexyl)amino]carbonyl}benzoate;
N-(trans-4-[2-(1,2,3,4-tetrahydro-1,4-epiazano-naphthalen-9-yl)-ethyl]-cyclohexyl)-2,2-diphenylpropanamide;
N-(trans-4-[2-(1,2,3,4-tetrahydro-1,4-epiazano-naphthalen-9-yl)-ethyl]-cyclohexyl)-4-[(phenylcarbonyl)amino]benzamide;
1-(trans-4-[2-(1,2,3,4-tetrahydro-1,4-epiazano-naphthalen-9-yl)-ethyl]-cyclohexyl)-3-(2,2-diphenylethyl)urea;
N′-(trans-4-[2-(1,2,3,4-tetrahydro-1,4-epiazano-naphthalen-9-yl)-ethyl]-cyclohexyl)-N,1-bis(phenylmethyl)urea;
1-(trans-4-[2-(1,2,3,4-tetrahydro-1,4-epiazano-naphthalen-9-yl)-ethyl]-cyclohexyl)-3-(3,3-diphenylpropyl)urea;
1-Benzyl-3-{4-[2-(1,2,3,4-tetrahydro-1,4-epiazano-naphthalen-9-yl)-ethyl]-cyclohexyl}-urea
1-(1-Naphthalen-1-yl-ethyl)-3-{4-[2-(1,2,3,4-tetrahydro-1,4-epiazano-naphthalen-9-yl)-ethyl]-cyclohexyl}-urea.

3. A compound according to claim 1 consisting of the group selected from:
1-(trans-4-[2-(1,2,3,4-tetrahydro-1,4-epiazano-naphthalen-9-yl)-ethyl]-cyclohexyl)-2-(4-pyridinyl)acetamide;
1-(trans-4-[2-(1,2,3,4-tetrahydro-1,4-epiazano-naphthalen-9-yl)-ethyl]-cyclohexyl)-3-(2-pyridinylmethyl)urea;
1-(trans-4-[2-(1,2,3,4-tetrahydro-1,4-epiazano-naphthalen-9-yl)-ethyl]-cyclohexyl)-3-(4-hydroxycyclohexyl)urea;
N′-(trans-4-[2-(1,2,3,4-tetrahydro-1,4-epiazano-naphthalen-9-yl)-ethyl]-cyclohexyl)-N-methyl-N-(phenylmethyl)urea;
1-(trans-4-[2-(1,2,3,4-tetrahydro-1,4-epiazano-naphthalen-9-yl)-ethyl]-cyclohexyl)-3-[2-(2-pyridinyl)ethyl]urea;
N-(trans-4-[2-(1,2,3,4-tetrahydro-1,4-epiazano-naphthalen-9-yl)-ethyl]-cyclohexyl)-2-(2-pyrimidinylthio)acetamide;
N-(trans-4-[2-(1,2,3,4-tetrahydro-1,4-epiazano-naphthalen-9-yl)-ethyl]-cyclohexyl)-3-quinolinecarboxamide;
N-(trans-4-[2-(1,2,3,4-tetrahydro-1,4-epiazano-naphthalen-9-yl)-ethyl]-cyclohexyl)-1-methyl-1H-indole-2-carboxamide;
(2E)-N-(trans-4-[2-(1,2,3,4-tetrahydro-1,4-epiazano-naphthalen-9-yl)-ethyl]-cyclohexyl)-4-oxo-4-phenyl-2-butenamide;
1-(trans-4-[2-(1,2,3,4-tetrahydro-1,4-epiazano-naphthalen-9-yl)-ethyl]-cyclohexyl)-3-(1H-indol-3-ylmethyl)urea;
1-(trans-4-[2-(1,2,3,4-tetrahydro-1,4-epiazano-naphthalen-9-yl)-ethyl]-cyclohexyl)-3-(1H-benzimidazol-2-ylmethyl)urea;
1-(trans-4-[2-(1,2,3,4-tetrahydro-1,4-epiazano-naphthalen-9-yl)-ethyl]-cyclohexyl)-3-(1,2,3,4-tetrahydro-2-naphthalenyl)urea;
1-(trans-4-[2-(1,2,3,4-tetrahydro-1,4-epiazano-naphthalen-9-yl)-ethyl]-cyclohexyl)-3-(1,2,3,4-tetrahydro-1-naphthalenyl)urea;
1-(trans-4-[2-(1,2,3,4-tetrahydro-1,4-epiazano-naphthalen-9-yl)-ethyl]-cyclohexyl)-N,N-dimethylphenylalaninamide;
1-(trans-4-[2-(1,2,3,4-tetrahydro-1,4-epiazano-naphthalen-9-yl)-ethyl]-cyclohexyl)-3-(4-phenylbutyl)urea;
1-(trans-4-[2-(1,2,3,4-tetrahydro-1,4-epiazano-naphthalen-9-yl)-ethyl]-cyclohexyl)-3-(2-methyl-1,2,3,4-tetrahydro-2-naphthalenyl)urea;
1-(trans-4-[2-(1,2,3,4-tetrahydro-1,4-epiazano-naphthalen-9-yl)-ethyl]-cyclohexyl)-3-(2-methyl-1,2,3,4-tetrahydro-2-naphthalenyl)urea;
N-(trans-4-[2-(1,2,3,4-tetrahydro-1,4-epiazano-naphthalen-9-yl)-ethyl]-cyclohexyl)-4-(2-pyridinyl)-1-piperazinecarboxamide;
1-(trans-4-[2-(1,2,3,4-tetrahydro-1,4-epiazano-naphthalen-9-yl)-ethyl]-cyclohexyl)-3-[2-(4-pyridinyl)ethyl]urea formate;
N-(trans-4-[2-(1,2,3,4-tetrahydro-1,4-epiazano-naphthalen-9-yl)-ethyl]-cyclohexyl)-2,2-diphenylacetamide;
N-(trans-4-[2-(1,2,3,4-tetrahydro-1,4-epiazano-naphthalen-9-yl)-ethyl]-cyclohexyl)-2,2-diphenylacetamide;
1-(trans-4-[2-(1,2,3,4-tetrahydro-1,4-epiazano-naphthalen-9-yl)-ethyl]-cyclohexyl)-3-[3-(1H-imidazol-1-yl)propyl]urea formate;
1-(trans-4-[2-(1,2,3,4-tetrahydro-1,4-epiazano-naphthalen-9-yl)-ethyl]-cyclohexyl)-3-{[4-(trifluoromethyl)phenyl]methyl}urea;
N-(trans-4-[2-(1,2,3,4-tetrahydro-1,4-epiazano-naphthalen-9-yl)-ethyl]-cyclohexyl)-4-(phenylmethyl)-1-piperazinecarboxamide;
N-{5-[(trans-4-[2-(1,2,3,4-tetrahydro-1,4-epiazano-naphthalen-9-yl)-ethyl]-cyclohexyl)amino]-5-oxopentyl}benzamide;
1-(trans-4-[2-(1,2,3,4-tetrahydro-1,4-epiazano-naphthalen-9-yl)-ethyl]-cyclohexyl)-3-(1H-indol-3-ylmethyl)urea formate;
1-(trans-4-[2-(1,2,3,4-tetrahydro-1,4-epiazano-naphthalen-9-yl)-ethyl]-cyclohexyl)-3-{[3-(dimethylamino)phenyl]methyl}urea formate;
N-(trans-4-[2-(1,2,3,4-tetrahydro-1,4-epiazano-naphthalen-9-yl)-ethyl]-cyclohexyl)-3-(4-methylphenyl)-3-phenylpropanamide;
N-(trans-4-[2-(1,2,3,4-tetrahydro-1,4-epiazano-naphthalen-9-yl)-ethyl]-cyclohexyl)-4,4-diphenylbutanamide;
1-(trans-4-[2-(1,2,3,4-tetrahydro-1,4-epiazano-naphthalen-9-yl)-ethyl]-cyclohexyl)-2-(methyloxy)-2,2-diphenylacetamide;
1-(trans-4-[2-(1,2,3,4-tetrahydro-1,4-epiazano-naphthalen-9-yl)-ethyl]-cyclohexyl)-3-(1-naphthalenylmethyl)urea formate;
N-(trans-4-[2-(1,2,3,4-tetrahydro-1,4-epiazano-naphthalen-9-yl)-ethyl]-cyclohexyl)-4-(phenylmethyl)-1-piperazinecarboxamide formate;
1-(trans-4-[2-(1,2,3,4-tetrahydro-1,4-epiazano-naphthalen-9-yl)-ethyl]-cyclohexyl)-3-(2-{3-[hydroxy(3-pyridinyl)methyl]phenyl}ethyl)urea;
1-(trans-4-[2-(1,2,3,4-tetrahydro-1,4-epiazano-naphthalen-9-yl)-ethyl]-cyclohexyl)-3-[1-(phenylmethyl)-4-piperidinyl]urea formate;
1-(trans-4-[2-(1,2,3,4-tetrahydro-1,4-epiazano-naphthalen-9-yl)-ethyl]-cyclohexyl)-3-(3-phenylpropyl)urea trifluoroacetate;
1-(trans-4-[2-(1,2,3,4-tetrahydro-1,4-epiazano-naphthalen-9-yl)-ethyl]-cyclohexyl)-3-[5,8-bis(methyloxy)-1,2,3,4-tetrahydro-2-naphthalenyl]urea formate;
N′-(trans-4-[2-(1,2,3,4-tetrahydro-1,4-epiazano-naphthalen-9-yl)-ethyl]-cyclohexyl)-N-(3,3-diphenylpropyl)-N-propylurea;
1-(trans-4-[2-(1,2,3,4-tetrahydro-1,4-epiazano-naphthalen-9-yl)-ethyl]-cyclohexyl)-3-(3,3-diphenylpropyl)urea formate;
1-(trans-4-[2-(1,2,3,4-tetrahydro-1,4-epiazano-naphthalen-9-yl)-ethyl]-cyclohexyl)-3-(1-methyl-2,2-diphenylethyl)urea formate;
N-(trans-4-[2-(1,2,3,4-tetrahydro-1,4-epiazano-naphthalen-9-yl)-ethyl]-cyclohexyl)-2-[(2-methylphenyl)(phenyl)methyl]benzamide;
N-(trans-4-[2-(1,2,3,4-tetrahydro-1,4-epiazano-naphthalen-9-yl)-ethyl]-cyclohexyl)-4-(diethylamino)-2,2-diphenylbutanamide;
1-(trans-4-[2-(1,2,3,4-tetrahydro-1,4-epiazano-naphthalen-9-yl)-ethyl]-cyclohexyl)-3-(2-{3-[hydroxy(3-pyridinyl)methyl]phenyl}ethyl)urea formate;
1-(trans-4-[2-(1,2,3,4-tetrahydro-1,4-epiazano-naphthalen-9-yl)-ethyl]-cyclohexyl)-3-(1,1-dimethyl-3,3-diphenylpropyl)urea formate;
N′-(trans-4-[2-(1,2,3,4-tetrahydro-1,4-epiazano-naphthalen-9-yl)-ethyl]-cyclohexyl)-N-(3,3-diphenylpropyl)-N-ethylurea formate;
N′-(trans-4-[2-(1,2,3,4-tetrahydro-1,4-epiazano-naphthalen-9-yl)-ethyl]-cyclohexyl)-N-methyl-N-(2,2,2-triphenylethyl)urea;
N-(trans-4-[2-(1,2,3,4-tetrahydro-1,4-epiazano-naphthalen-9-yl)-ethyl]-cyclohexyl)-3-phenyl-3-{3-[(phenylmethyl)oxy]phenyl}propanamide;
N-(trans-4-[2-(1,2,3,4-tetrahydro-1,4-epiazano-naphthalen-9-yl)-ethyl]-cyclohexyl)-2-hydroxy-2,2-diphenylacetamide trifluoroacetate;
N′-(trans-4-[2-(1,2,3,4-tetrahydro-1,4-epiazano-naphthalen-9-yl)-ethyl]-cyclohexyl)-N-ethyl-N-(3-hydroxy-3,3-diphenylpropyl)urea formate;
N-(trans-4-[2-(1,2,3,4-tetrahydro-1,4-epiazano-naphthalen-9-yl)-ethyl]-cyclohexyl)-2-{bis[4-(dimethylamino)phenyl]methyl}benzamide;
N-(trans-4-[2-(1,2,3,4-tetrahydro-1,4-epiazano-naphthalen-9-yl)-ethyl]-cyclohexyl)-3-[4-(dimethylamino)phenyl]-3-phenylpropanamide trifluoroacetate;
N′-(trans-4-[2-(1,2,3,4-tetrahydro-1,4-epiazano-naphthalen-9-yl)-ethyl]-cyclohexyl)-N-(3,3-diphenylpropyl)-N-(phenylmethyl)urea formate;
N-(trans-4-[2-(1,2,3,4-tetrahydro-1,4-epiazano-naphthalen-9-yl)-ethyl]-cyclohexyl)-2,2-bis(4-chlorophenyl)acetamide trifluoroacetate;
N-(trans-4-[2-(1,2,3,4-tetrahydro-1,4-epiazano-naphthalen-9-yl)-ethyl]-cyclohexyl)-4-(diethylamino)-2,2-diphenylbutanamide trifluoroacetate;
1-(trans-4-[2-(1,2,3,4-tetrahydro-1,4-epiazano-naphthalen-9-yl)-ethyl]-cyclohexyl)-3-[3-(4-biphenylyl)-3-(4-chlorophenyl)-3-hydroxypropyl]urea formate;
1-(4-Bromo-benzyl)-3-{4-[2-(1,2,3,4-tetrahydro-1,4-epiazano-naphthalen-9-yl)-ethyl]-cyclohexyl}-urea;
1-(1,1-Diphenyl-methyl)-3-{4-[2-(1,2,3,4-tetrahydro-1,4-epiazano-naphthalen-9-yl)-ethyl]-cyclohexyl}-urea;
1-(2-Methoxy-benzyl)-3-{4-[2-(1,2,3,4-tetrahydro-1,4-epiazano-naphthalen-9-yl)-ethyl]-cyclohexyl}-urea;
1-(3-Methoxy-benzyl)-3-{4-[2-(1,2,3,4-tetrahydro-1,4-epiazano-naphthalen-9-yl)-ethyl]-cyclohexyl}-urea;
1-(4-Methoxy-benzyl)-3-{4-[2-(1,2,3,4-tetrahydro-1,4-epiazano-naphthalen-9-yl)-ethyl]-cyclohexyl)-urea;
2-Methyl-quinoline-5-carboxylic acid {4-[2-(1,4-dihydro-1,4-epiazano-naphthalen-9-yl)-ethyl]-cyclohexyl}-amide;
8-Chloro-2-methyl-quinoline-5-carboxylic acid {4-[2-(1,4-dihydro-1,4-epiazano-naphthalen-9-yl)-ethyl]-cyclohexyl}-amide;
8-Methoxy-2-methyl-quinoline-5-carboxylic acid {4-[2-(1,4-dihydro-1,4-epiazano-naphthalen-9-yl)-ethyl]-cyclohexyl}-amide;
Quinoxaline-5-carboxylic acid {4-[2-(1,4-dihydro-1,4-epiazano-naphthalen-9-yl)-ethyl]-cyclohexyl}-amide;
Quinoline-5-carboxylic acid {trans-4-[1-(1,2,3,4-tetrahydro-1,4-epiazano-naphthalen-9-yl)methyl]-cyclohexylmethyl}-amide;
8-Methyl-quinoline-5-carboxylic acid {trans-4-[1-(1,2,3,4-tetrahydro-1,4-epiazano-naphthalen-9-yl)methyl]-cyclohexylmethyl}-amide;
2-Methyl-quinoline-5-carboxylic acid {trans-4-[(1S,4S)-1-(1,2,3,4-tetrahydro-1,4-epiazano-naphthalen-9-yl) methyl]-cyclohexylmethyl}-amide;
8-Chloro-2-methyl-quinoline-5-carboxylic acid {trans-4-[1-(1,2,3,4-tetrahydro-1,4-epiazano-naphthalen-9-yl)methyl]-cyclohexylmethyl}-amide;
2,8-Dimethyl-quinoline-5-carboxylic acid {trans-4-[1-(1,2,3,4-tetrahydro-1,4-epiazano-naphthalen-9-yl)methyl]-cyclohexylmethyl}-amide;
1-((S)-1-Naphthalen-1-yl-ethyl)-3-{trans-4-[1-(1,2,3,4-tetrahydro-1,4-epiazano-naphthalen-9-yl)methyl]-cyclohexylmethyl}-urea;
1-((R)-1-Naphthalen-1-yl-ethyl)-3-{trans-4-[(1-(1,2,3,4-tetrahydro-1,4-epiazano-naphthalen-9-yl)methyl]-cyclohexylmethyl}-urea;
Isoquinoline-1-carboxylic acid {trans-4-[(1S,4R)-2-(1,2,3,4-tetrahydro-1,4-epiazano-naphthalen-9-yl)-ethyl]-cyclohexyl}-amide;
Acridine-9-carboxylic acid {trans-4-[2-(1,2,3,4-tetrahydro-1,4-epiazano-naphthalen-9-yl)-ethyl]-cyclohexyl}-amide;
2,3-Dihydro-naphthalene-1-carboxylic acid {trans-4-[2-(1,2,3,4-tetrahydro-1,4-epiazano-naphthalen-9-yl)-ethyl]-cyclohexyl}-amide;
6,7-Dihydro-quinoline-8-carboxylic acid {trans-4-[2-(1,2,3,4-tetrahydro-1,4-epiazano-naphthalen-9-yl)-ethyl]-cyclohexyl}-amide;
9-[2-(trans-4-{[1-(2-Methyl-quinolin-5-yl)-methanoyl]-amino}-cyclohexyl)-ethyl]-1,4-dihydro-1,4-epiazano-naphthalene-6-carboxylic acid methyl ester;
9-(2-{trans-4-[3-((S)-1-Naphthalen-1-yl-ethyl)-ureido]-cyclohexyl}-ethyl)-1,4-dihydro-1,4-epiazano-naphthalene-6-carboxylic acid methyl ester;
9-[2-(trans-4-{[1-(2-Methyl-quinolin-5-yl)-methanoyl]-amino)-cyclohexyl)-ethyl]-1,2,3,4-tetrahydro-1,4-epiazano-naphthalene-6-carboxylic acid methyl ester;
(1S,4R)-9-(2-(4-[(1-Quinolin-5-yl-methanoyl)-amino]-cyclohexyl}-ethyl)-1,2,3,4-tetrahydro-1,4-epiazano-naphthalene-6-carboxylic acid methyl ester;
9-(2-{trans-4-[3-((S)-1-Naphthalen-1-yl-ethyl)-ureido]-cyclohexyl}-ethyl)-1,2,3,4-tetrahydro-1,4-epiazano-naphthalene-6-carboxylic acid methyl ester;
1-(trans-4-{2-[6-(2-Methyl-propanoyl)-1,2,3,4-tetrahydro-1,4-epiazano-naphthalen-9-yl]-ethyl}-cyclohexyl)-3-((S)-1-naphthalen-1-yl-ethyl)-urea;
Quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
8-Chloro-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
8-Chloro-2-methyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
8-Methyl-quinoline-5-carboxylic acid (4-{2-[(1S,4R)-6-(2-methyl-propanoyl)-1,2,3,4-tetrahydro-1,4-epiazano-naphthalen-9-yl]-ethyl}-cyclohexyl)-amide;
2,8-Dimethyl-quinoline-5-carboxylic acid (trans-4-{2-[6-(2-methyl-propanoyl)-1,2,3,4-tetrahydro-1,4-epiazano-naphthalen-9-yl]-ethyl}-cyclohexyl)-amide;
1-(trans-4-{2-[6-(2-Methyl-propanoyl)-1,2,3,4-tetrahydro-1,4-epiazano-naphthalen-9-yl]-ethyl}-cyclohexyl)-3-((R)-1-naphthalen-1-yl-ethyl)-urea;
1-(trans-4-{2-[6-Butyryl-1,2,3,4-tetrahydro-1,4-epiazano-naphthalen-9-yl]-ethyl}-cyclohexyl)-3-((S)-1-naphthalen-1-yl-ethyl)-urea;
Quinoline-5-carboxylic acid (trans-4-{2-[6-butyryl-propanoyl)-1,2,3,4-tetrahydro-1,4-epiazano-naphthalen-9-yl]-ethyl}-cyclohexyl)-amide;
8-Chloro-2-methyl-quinoline-5-carboxylic acid (trans-4-{2-[6-butyryl-1,2,3,4-tetrahydro-1,4-epiazano-naphthalen-9-yl]-ethyl}-cyclohexyl)-amide;
Quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
1-((S)-1-Naphthalen-1-yl-ethyl)-3-(trans-4-{2-[6-(2-phenyl-ethanoyl)-1,2,3,4-tetrahydro-1,4-epiazano-naphthalen-9-yl]-ethyl}-cyclohexyl)-urea;
8-Methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
2,8-Dimethyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
8-Methoxy-2-methyl-quinoline-5-carboxylic acid (trans-4-{2-[6-(2-phenyl-ethanoyl)-1,2,3,4-tetrahydro-1,4-epiazano-naphthalen-9-yl]-ethyl}-cyclohexyl)-amide;
1-((R)-1-Naphthalen-1-yl-ethyl)-3-(4-{2-[(1S,4R)-6-(2-phenyl-ethanoyl)-1,2,3,4-tetrahydro-1,4-epiazano-naphthalen-9-yl]-ethyl}-cyclohexyl)-urea;
Quinoline-5-carboxylic acid methyl-{trans-4-[1-(1,2,3,4-tetrahydro-1,4-epiazano-naphthalen-9-yl)methyl]-cyclohexylmethyl}-amide8-Methyl-quinoline-5-carboxylic acid methyl-{trans-4-[1-(1,2,3,4-tetrahydro-1,4-epiazano-naphthalen-9-yl)methyl]-cyclohexylmethyl}-amide;
2,8-Dimethyl-quinoline-5-carboxylic acid methyl-{trans-4-[1-(1,2,3,4-tetrahydro-1,4-epiazano-naphthalen-9-yl)methyl]-cyclohexylmethyl}-amide;
8-Chloro-quinoline-5-carboxylic acid methyl-{4-[(1S,4S)-1-(1,2,3,4-tetrahydro-1,4-epiazano-naphthalen-9-yl)methyl]-cyclohexylmethyl}-amide;
8-Chloro-2-methyl-quinoline-5-carboxylic acid methyl-(trans-4-[1-(1,2,3,4-tetrahydro-1,4-epiazano-naphthalen-9-yl)methyl]-cyclohexylmethyl}-amide and pharmaceutically acceptable salts thereof.

4. A pharmaceutical composition for the treatment of muscarinic acetylcholine receptor mediated diseases comprising a compound according to claim 1 and a pharmaceutically acceptable carrier thereof.

5. A method of inhibiting the binding of acetylcholine to its receptors in a mammal in need thereof comprising administering a safe and effective amount of a compound according to claim 1.

6. A method of treating a muscarinic acetylcholine receptor mediated disease, wherein acetylcholine binds to said receptor, comprising administering a safe and effective amount of a compound according to claim 1.

7. A method according to claim 6 wherein the disease is selected from the group consisting of chronic obstructive lung disease, chronic bronchitis, asthma, chronic respiratory obstruction, pulmonary fibrosis, pulmonary emphysema and allergic rhinitis.

8. A method according to claim 7 wherein administration is via inhalation via the mouth or nose.

9. A method according to claim 8 wherein administration is via a medicament dispenser selected from a reservoir dry powder inhaler, a multi-dose dry powder inhaler or a metered dose inhaler.

10. A method according to claim 9 wherein the compound is administered to a human and has a duration of action of 12 hours or more for a 1 mg dose.

11. A method according to claim 10 wherein the compound has a duration of action of 24 hours or more.

12. A method according to claim 11 wherein the compound has a duration of action of 36 hours or more.
